(19) United States
(12) Patent Application Publication
(10) Pub. No.: US 2014/0127159 A1
(43) Pub. Date: May 8, 2014

(54) HCV Combination Therapy

(71) Applicant: Michael Hodges, San Diego, CA (US)

(72) Inventor: Michael Hodges, San Diego, CA (US)

(73) Assignee: Stella ApS, Hørsholm (DK)

(21) Appl. No.: 14/128,504

(22) PCT Filed: Jun. 25, 2012
(86) PCT No.: PCT/EP2012/062205

§ 371 Date: Dec. 20, 2013

Related U.S. Application Data
(60) Provisional application No. 61/500,144, filed on Jun. 23, 2011.

(51) Int. Cl. A61K 31/713; A61K 38/21; A61K 38/07; A61K 31/7068; A61K 31/4178; A61K 31/381; A61K 31/7056

(52) U.S. Cl. CPC A61K 31/713; A61K 31/381; A61K 38/212; A61K 31/7056; A61K 31/7068; A61K 31/4178; A61K 38/07
USPC 424/85.7; 514/44 A; 514/4.3

(57) ABSTRACT

The present invention relates to the treatment of hepatitis C (HCV) infection by the combination treatment with a miR-122 inhibitor and a HCV NS3/4A protease inhibitor.

### RELATED APPLICATIONS

This application claims priority from US provisional applications 61/500144 filed 23 Jun. 2011, which is hereby incorporated by reference.

### FIELD OF INVENTION

The present invention relates to the treatment of hepatitis C (HCV) infection by the combination treatment with a miR-122 inhibitor and a HCV NS3/4A protease inhibitor.

### BACKGROUND

Hepatitis C virus (HCV) infections affect approximately 3 percent of the worldwide population and often lead to cirrhosis and hepatocellular carcinoma. The standard therapy of pegylated-interferon and ribavirin induces serious side effects and provides viral eradication in less than 50% of patients. Combination therapy of HCV including ribavirin and interferonare currently is the approved therapy for HCV. Unfortunately, such combination therapy also produces side effects and is often poorly tolerated, resulting in major clinical challenges in a significant proportion of patients. Numerous direct acting agents (DAAs) have been or are being developed for treatment of HCV, such as telaprevir and boceprevir (both received ma approved in 2011 for use with interferon and ribavirin based therapy), however direct acting agents are linked to increased toxicity of treatment, the emergence of resistance, and to date do not provide a standard of care which is interferon free. The combination of direct acting agents can also result in drug-drug interactions. To date, no HCV therapy has been approved which is interferon free. There is therefore a need for new combination therapies which have reduced side effects, and interferon free, have a reduced emergence of resistance, reduced treatment periods and/or and enhanced cure rates.

### SUMMARY OF INVENTION

The invention refers to the therapeutic use of microRNA-122 inhibitors, such as miravirsen, in combination with at least one further anti-viral compound, such as a NS3/4A polymerse inhibitor and/or ribavirin. The combination treatment may, in some embodiments be interferon free.

The invention provides for a method for the treatment of hepatitis C (HCV) infection in a subject infected with HCV, said method comprising the steps of administering a miR-122 inhibitor (also referred to herein as a miR-122 antagonist) and a HCV NS3/4A protease inhibitor to the subject infected with HCV.

The treatment may, optionally further include the step of administering ribavirin, or a virally active derivative thereof, to the subject. The treatment may, in some embodiments be interferon free.

The invention provides for a method of reducing the level of HCV infection in a cell, said method comprising contacting a cell infected with a HCV with a miR-122 inhibitor and at least one HCV NS3/4A protease inhibitor. The method may, optionally, further include the step of administering ribavirin, or a virally active derivative thereof, to the cell.

The invention provides for the use of a miR-122 inhibitor for the preparation of a medicament for the treatment of Hepatitis C, wherein said medicament is for use in combination with an HCV NS3/4A protease inhibitor.

The invention provides for a miR-122 inhibitor for use in the treatment of Hepatitis C in combination with an HCV NS3/4A protease inhibitor.

Optionally, the use may also be in combination with ribavirin, or a virally active derivative thereof. In some embodiments, the use may be interferon free.

The miR-122 inhibitor may, in some embodiments, be an oligomer which is complementary to the has-miR-122 microRNA sequence across the oligomer (an antisense oligomer), such as miravirsen (SPC3649):

5′-^{m}Cₛ^{o}cₛAₛ^{o}tₛtₛGₛ^{o}Tₛ^{o}cₛaₛ^{m}Cₛ^{o}aₛ^{m}Cₛ^{o}tₛ^{m}Cₛ^{om}C^{o}-3′

The HCV NS3/4A protease inhibitor may, in some embodiments, including but not limited to when the miR-122 inhibitor is an antisense oligomer, such as SPC3649), be selected from the groups consisting of Tegobuvir, Narlaprevir, Vaniprevir, Danoprevir, Filibuvir, Telaprevir, Coceprevir and Boceprevir; or one of the other HCV NS3/4A protease inhibitors referred to herein.

The HCV NS3/4A protease inhibitor may, in some embodiments, including but not limited to when the miR-122 inhibitor is an antisense oligomer, such as SPC3649), be selected from the groups consisting of ABT-450 (Abbott/Enanta), ACH-2684; ACH-1625 (Achillion), BMS-650032 (BMS); BMS-791325 (BMS), TMC435 (Tibotec), GS-9256 (Gilead), RG7227 (Intermune/Genetech), MK-5172 (Merck); MK-7009 (Merck), and BI 201335 (Boehringer Ingleheim Pharma).

Suitably, the miR-122 inhibitor may be administered in an effective amount. Suitably, the HCV NS3/4A protease inhibitor may be administered in an effective amount. Suitably ribarivin, of the virally active derivative thereof, when used, may be used in an effective amount.

### FIGURES

FIG. 1: Schematic of some combination treatment regimens, with an optional pre-treatment period of either compound 1 or compound 2, optionally in the presence of compound 3 and optionally 4, or in the absence of compound 4. The combination pre-tretament period refers to a period of between 4 and 48 weeks wherein compound 1 is administered in conjunction with compound 2, optionally in combination with a further agent, compound 3 and optionally compound 4. There is an optionally post treatment period with compound 3 and optionally compound 4.

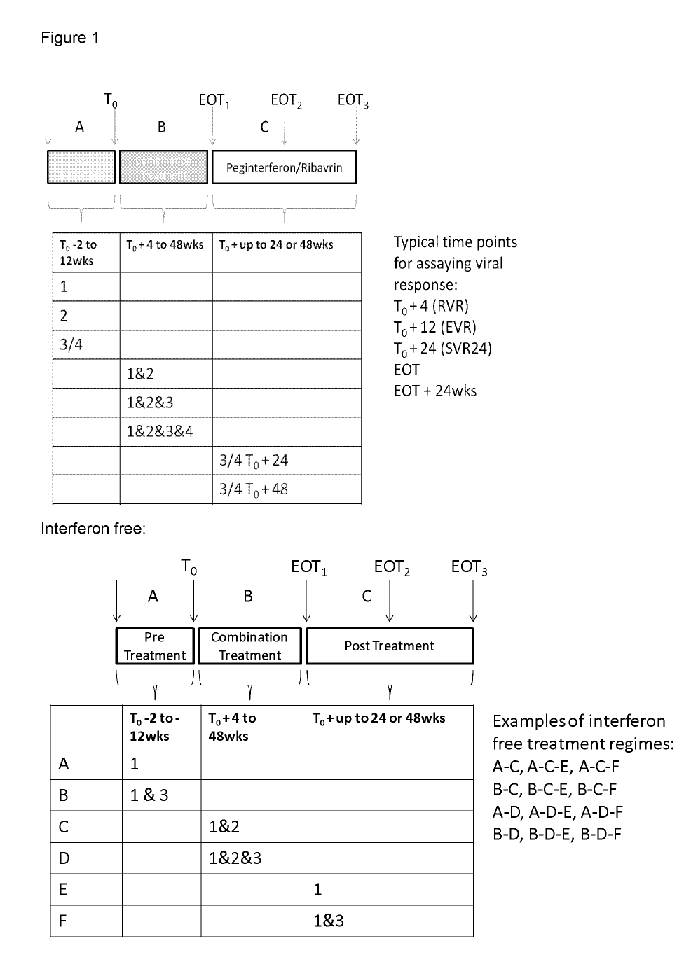

FIG. 2: Cells cultured were cultured in the in the presence of G418 and the indicated concentration of SPC4729, SPC3649, or telaprevir, or cell culture medium alone with G418 for 28 days were stained with crystal violet.

### DETAILED DESCRIPTION OF INVENTION

### Combination Treatments

Compound 1: The miR-122 inhibitor, such as the antisense oligomer.
Compound 2: An NS3a protease inhibitor or an HCV NS3/4A protease inhibitor (these terms are used interchangeably herein).
Compound 3: Ribavirin, or a virally active derivative thereof.
Compound 4: Interferon.
Compound 5: A further direct acting agent.
   The invention relates to a combination treatment comprises administering at least compound 1 and 2 to a subject infected with HCV.

Suitably, the combination treatment comprises administering at least compound 1 and 2 to a subject infected with HCV, and optionally compound 3 to a subject infected with HCV, wherein, in some embodiments, the combination treatment is interferon free—i.e. during the combination treatment period, compound 4 is not administered to the subject.

In some embodiments, the combination treatment which administering compound 1, 2 and 3 to a subject infected with HCV.

In some embodiments, the combination treatment which administering compound 1, 2, 3 and 4 to a subject infected with HCV.

In some embodiments, the combination treatment which administering compound 1 and 2 to a subject infected with HCV, wherein no compound 3 is administered to the subject during the combination treatment period.

In some embodiments, the combination treatment which administering compound 1 and 2 to a subject infected with HCV, wherein no compound 4 is administered to the subject during the combination treatment period.

In some embodiments, the combination treatment which administering compound 1 and 2 to a subject infected with HCV, wherein no compound 3 and no compound 4 is administered to the subject during the combination treatment period.

In the above embodiments, optionally compound 5 may also be administered. Clinical trials are either being planned or are underway for the combined use of compound 1 and compound 5, typically in combination with compound 3. Compound 5 may be a HCV NS5B polymerase inhibitor, such as a non-nucleoside or a nucleoside HCV NS5B polymerase inhibitor, and/or a HCV NS5A protein inhibitor. In some embodiments, a compound 5 is not administered during the treatment period.

Ritonavir; 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate, is a CYP3A4 inhibitor which is often used in combinations with direct acting agents, and may be used in the combination therapy of the present invention, such as during the combination treatment period. It is thought that ritonavir inhibits the cytochrome P450, in particular CYP3A mediated metabolism of some DAAs, enhancing the amount of drug in the blood and subsequently the efficacy of the DAA. In some embodiments, an inhibitor of cytochrome P450 is administered during the combination treatment period.

Two or more combined compounds may be used together or sequentially, i.e. one compound used in the method of the invention may be used prior to, during or subsequent to one or more of the other therapeutic agents referred to in the method of the invention (a combination treatment). The combined use of both (or more) agents suitably overlap so that the therapeutic effect of one agent (i.e. the time period post use where a measurable benefit to the patient is observed), is concurrent, at least at some point, with the period of therapeutic effect of a second agent. In some embodiments, the combined use of compounds 1 and 2 and optionally 3 are concurrent, and the concurrent combination treatment may optionally be followed, with treatment of compounds 3 and/or 4. For example, in some embodiments, the administration of compound 4 may be subsequent to the combination treatment period. In some embodiments, the administration of compound 4 may be subsequent to the final dose of compounds 1 and/or 2. In some embodiments, the subsequent administration of compound 4 may be used in combination with compound 3.

In some embodiments, compound 1 and the compound 2 are used either together or sequentially. In some embodiments, the administration of at least one administration of compound 1, and at least one administration of compound 2, may be on the same day, or within the same week, or within the same 2 weeks, or within three or four weeks of the administration of the antisense oligomer. In some embodiments, compound 3 may also be used together or sequentially along with the compound 1 and compound 2, such as on the same day, or within the same week, or within the same 2 weeks, or within three or four weeks of the administration of compound 1 and/or compound 2.

In some embodiments, such a double (compounds 1 and 2) or triple (compounds 1, 2 and 3) (compounds 1, 2, and 5) (compounds 1, 2, 3 and 5) combination therapies do not include the administration of compound 4 (an interferon, such as pegylated interferon alfa-2a) during the combination treatment period.

The combination treatment period may be at least 4 weeks, such as at least 8 weeks, such as at least 12 weeks, such as at least 16 weeks, such as at least 24 weeks, from the time when the first combination treatment has been administered, i.e. the time from when the subject has been administered at least both compounds 1 and 2. In some embodiments, the combination treatment period may be up to 6 months in duration. Multiple administrations of each of compound 1, compound 2 and optionally compound 3 and/or compound 5 are typically administered during the combination treatment period. In some other embodiments, compound 4 may also administered during the combination period although it is recognised that interferon free treatments are preferable.

Alternatively, or in addition, compound 4 may be administered after the combination period, such as for at least about 8 weeks, such as for at least about 8 weeks, such as at least about 12 weeks, such as for at least about 16 weeks, such as at least about 24 weeks, such as up to about 24 weeks or up to about 48 weeks. Typically if compound 4 is administered, it is administered with compound 3.

The invention further provides for the a method for the treatment of hepatitis C (HCV) infection in a subject infected with HCV, said method comprising the steps of administering compound 1 (also referred to herein as a miR-122 antagonist, e.g. miravirsen) and compound 3, e.g. ribavirin, or a virally active derivative thereof, to the subject. The treatment may, in some embodiments be interferon free. In some embodiments, said method may or may not comprise the administration of compound 2. In this regard, the results provided herein, and the result obtained from miravirsen monotherapy in phase 2 clinical trials, indicate that the combined use of compound 1, such as miravirsen in combination with ribavirin, as disclosed herein, appears sufficient to effectively treat or cure HCV infection, even in the absence of another anti-viral therapeutic, for example compound 4, or compound 2, such as a HCV NS5A protein inhibitor, and/or a HCV NS5B polymerase inhibitor and/or a HCV NS3/4A protease inhibitor. The combined use of miravirsen and ribavirin may be as described herein in connection with a DAA/miravirsen. The invention provides for a miR-122 inhibitor, e.g. miravirsen for use in the treatment of Hepatitis C in combination with compound 3, e.g. ribavirin. The invention provides for the use of a miR-122 inhibitor for the preparation of a medicament for the treatment of Hepatitis C, wherein said medicament is for use in combination with compound 3, e.g. ribavirin.

In some embodiments the combination treatment period is about 4 weeks or about 8 weeks, or about 12 weeks. In some embodiments the combination treatment period is a minimum of about 4 weeks, or a minimum of about 8 weeks, or a minimum of about 12 weeks. In some embodiments, the maximum period of combination treatment is about 24 weeks, about 36 weeks or about 48 weeks. In some embodiments, the combination treatment period is about 4-about 8 weeks, about 4-about 12 weeks, about 4-about 24 weeks, about 4-about 36 weeks, about 4-about 48 weeks, about 8-about 12 weeks, about 8-about 24 weeks, about 8-about 36 weeks, about 8-about 48 weeks, about 12-about 24 weeks, about 12-about 36 weeks, about 12-about 48 weeks, about 24-about 36 weeks, about 24-about 48 weeks, in duration.

During the combination treatment period at least a dose of compound 1 and a dose of compound 2 are administered to the subject (patient).

In some embodiments, compound 2 may be administered prior to the start of the combination treatment period (i.e. a pre treatment/lead in treatment), such as for a period of about 2-about 12 weeks, such as about 4 weeks prior to the start of the combination treatment period. In some embodiments compound 2 is bocepravir, which is used as a pre-treatment.

The emergence of viral resistance to direct acting agents is a major limitation in the successful development and application of HCV therapeutics, including treatment therapies which are interferon free. HCV replicates by using HCV-RNA-dependent RNA-polymerase NS5B which has poor fidelity and lacks proof reading activity. Therefore, HCV is associated with a high frequency of errors in copying the HCV genome. In addition, HCV has a high turnover rate, hence, a patient with chronic HCV infection (CHC) will be infected with multiple HCV “quasi” species. Some of these species, called Resistant Associate Variants (RAVs), will have a lower susceptibility and/or resistance to direct acting antiviral agents (DAAs). When DAAs are given to patients with CHC there will be a reduction in the quasi species with high susceptibility to the DAAs. The RAVs will now dominate the pool of HCV quasi species and are able to infect the un-infected hepatocytes. This so called “expansion in to the replication space” is the cause of virological failure, which occurs rapidly (virological breakthrough seen within days of starting DAA monotherapy) when CHC is treated with DAA monotherapy. Virologicfailure (virologic breakthrough or relapse seen after initial clearance of HCV RNA from blood) can also occur when the multiple DAAs are given in combination with or without peg-IFN and ribavirin. Miravirsen sequesters miR-122, a micro RNA that is essential for HCV accumulation in the liver. Miravirsen distributes throughout the liver and will sequester miR-122, thus preventing HCV from using miR-122. Consequently, when MIR treatment is given either prior to and/or together with DAA, the replication space will be protected from RAV expansion, and prevent virological failure.

In some embodiments, compound 1 is administered to the subject prior to compound 2, such as at least about 1 week, or at least about 2 weeks prior to administration of compound 2, such as at least about three weeks prior, such as at least about 4 weeks prior to the administration of compound 2. This may be referred to as the [compound 1] pre-treatment period and during the pre-treatment period, compound 1 may be administered alone, or, optionally in combination with compound 3. The pre-treatment period may, for example be up to about 12 weeks in duration—and as such may initiated e.g. about 2 weeks-about 12 weeks prior to the start of the combination treatment period. Typically the pre-treatment period allows for compound 1 to effectively reduce viral load in the subject prior to the administration of compound 2. This is useful in reducing or preventing the occurrence of resistance against compound 2. In some embodiments the pre-treatment period comprises of 1 or 2 doses of compound 1 (e.g.) miravirsen. Each (pre) dose of compound 1 may be, for example, dosed at about 5 mgs/kg, dosed at about 5 mgs/kg, or dosed at about 6 mgs/kg, or dosed at about 7 mgs/kg, or dosed at about 8 mgs/kg, or dosed at about 9 mgs/kg, or dosed at about 10 mgs/kg, or dosed at about 11 mgs/kg or at about 12 mgs/kg. In some embodiments a (e.g.single) pre-dose of compound 1 (e.g. miravirsen) is administered, e.g. at about or at least about 1 week, or at least about 2 weeks prior to, or at about or at least 3 weeks prior, or at about or at least 4 weeks prior to, the first administration of compound 2 or the combination treatment period. Suitably the (pre dose)(s) of compound 1 (e.g. miravirsen) are within about 12 weeks prior to the first administration of compound 2 or the combination treatment period, such as within about 10 or about 8 weeks prior to the first administration of compound 2 or the combination treatment period. The pre-treatment period may, in some embodiments further comprise the administration of compound 3, such as ribavirin to the subject.

Therefore, in an alternative embodiment, compound 1, such as miravirsen, may be administered prior to the start of the combination treatment period (i.e. a compound 1 pre treatment/lead in treatment), such as for a period of about 2 to about 24 weeks, such as about 4 weeks, or about 8 weeks or about 12 weeks, prior to the start of the combination treatment period. In some embodiments the pre-treatment period of compound 1 involves a series of administrations of compound 1 over the pre-treatment period with the aim of building up the concentration of compound 1 in the subject (such as in the liver of the subject) to a level which is therapeutically effective. As such, in some embodiments, the time interval between each administration of compound 1 during the pre-treatment period may be for example, selected from the group consisting of 1 day, 2 days, 3 days, 4 days, five days, six days and weekly. In some embodiments each dose of the pre-treatment period may, for example, be between about 0.1 mgs/kg and about 10 mgs/kg or 12 up to about 12 mgs/kg, such as about 0.2 mgs/kg, such as about 0.3 mgs/kg, such as about 0.4 mgs/kg, such as about 0.5 mgs/kg, such as about 0.6 mgs/kg, such as about 0.7 mgs/kg, such as about 0.8 mgs/kg, such as about 0.9 mgs/kg, such as about 1 mg/kg, such as about 2 mgs/kg, such as about 3 mgs/kg, such as about 4 mgs/kg, such as about 5 mgs/kg, such as about 6 mgs/kg, such as about 7 mgs/kg, such as about 8 mgs/kg, such as about 9 mgs/kg, such as about 10 mgs/kg, such as about 11 mgs/kg, such as about 12 mgs/kg. After the building-up phase, the administration of compound 1 may be as described herein, for example weekly, bi weekly or monthly. after the build up phase, a maintenance dosage could be given for a time period wherein the purpose is to maintain a relatively high activity or concentration of the compound in the target tissue, while e.g. the viral titre is decreased or other disease parameters are improved, after which the interval between each dosing could be increased or the dosage given at each dosing could be decreased or both, in order to maintain the disease at the new low level using the minimal needed effective dosage and at the same time obtain minimum side effects and the least inconvenience for the patient by having a high time interval in between administrations.

In some embodiments, after the build up phase, a maintenance dosage will be administered wherein the purpose is to maintain an effective concentration in the target tissue, in order to obtain the desired effect on important disease parameters, wherein the time interval in between each administration is large to avoid the inconvenience for the patient of the administration, and the dosage is kept to a minimum to avoid side effects while still maintaining the effect on the selected disease parameters.

In some embodiments, the build-up phase occurs during the pre-treatment period and the maintenance dose is administered as (the compound 1 part of) the combination-treatment period.

### Hepatitis C (HCV)

In some embodiments the subject has chronic hepatitis C (CHC). In some embodiments the subject has compensated cirrhosis. In some embodiments the subject cannot tolerate interferon (compound 4) treatment, such as the subject is contraindicated for interferon. In some embodiments the subject is a liver transplant patient. In some embodiments, the subject is co-infected with both HIV and HCV. In some embodiments the subject is an interferon non-responder. In some embodiments the subject has compensated liver disease. In some embodiments the subject has a history of failed treatment with interferon and/or ribavirin.

The development of DAAs has resulted in the identification of HCV subjects who are poor or non-responders to DAA treatment (DAA failures). In some embodiments, the subject is a subject who has been identified as a DAA failure, such as a subject who has responded poorly or has not responded to DAA treatment or has relapsed during or subsequent to DAA treatment. In this respect, in some embodiments, the DAA agent associated with the DAA failure is other than compound 2. In some embodiments the DAA associated with the DAA failure is selected from the group consisting of HCV NS5B polymerase inhibitors and/or HCV NS5A protein inhibitors. The NS5B polymerase inhibitors may, for example be, in some embodiments a non-nucleoside inhibitor or in some embodiments be a nucleoside inhibitor. In some embodiments the DAA failure is with a different NS3/4A protease inhibitor than the one used in the combination treatment of the present invention.

A significant population of patients with hepatitis C virus (HCV) respond poorly to the standard treatment with interferon. For example, non-responders and relapsers make up a large population of patients with hepatitis C virus (HCV) infection in the United States. For the purposes of the present invention, the term “non-responder” is, in some non-limiting embodiments, meant to refer to a HCV-infected subject, e.g., an HCV-infected primate, e.g., an HCV-infected human who does not exhibit a significant virologic response as a result of Interferon treatment, and that never becomes virus negative at any point during treatment. “Prior treatment” can involve, but is not limited to, any of the following hepatitis C antiviral regimens: standard interferon (IFN) monotherapy, standard IFN combination treatment with ribavirin (RBV), pegylated IFN alfa-2a monotherapy, pegylated IFN alfa-2b monotherapy, pegylated IFN alfa-2a combination therapy with RBV, pegylated IFN alfa-2b combination therapy with RBV. The term “slow responder” may refer to a HCV-infected subject, e.g., an HCV-infected primate, e.g., an HCV-infected human who does not develop a virologic response until about 24 weeks after the beginning of treatment with interferon therapy. “Partial responder” refers to a HCV-infected subject, e.g., an HCV-infected primate, e.g., an HCV-infected human who does not develop a virologic response until about 24 weeks after the beginning of treatment with interferon therapy, but the virologic response is not maintained at the end of the treatment. The term “partial Responder” refers to a patient who shows a greater than or equal to 2 log 10 reduction in HCV RNA at week 12, but not achieving HCV RNA undetectable at end of treatment with a Peg Interferon/RBV. The term “relapser” may refer to a HCV-infected subject, e.g., an HCV-infected primate, e.g., an HCV-infected human who has a virologic response that is HCV RNA negative and is maintained through the end of treatment, but relapse occurs before 6 months post-treatment. The terms “non-responder”, “slow responder”, “partial responder”, and “relapser” are not necessarily mutually exclusive. In some embodiments, the terms may be defined as below.

| Virological | | | EOT | 24 wks |
|---|---|---|---|---|
| Response | Week 4 | Week 12 | (24 or 48 wks) | post EOT |
| RVR | RNA −ve | | | |
| EVR (complete) | | RNA −ve | | |
| EVR (partial) | | ≧2 log fall | | |
| ETR | | | RNA −ve | |
| SVR | | | | RNA −ve |
| Null responder | | <2 log fall | | |
| Partial responder | | ≧2 log fall | RNA +ve | |
| Non-responder | | | RNA +ve | |
| Responder | | | RNA −ve | RNA +ve |
| Relapse | | | | |

RVR: Rapid Virological Response (may allow shortening of tx duration)
EVR: Early Virological Response (predicts lack of SVR)
ETR: End of Treatment Response
SVR: Sustained Virological Response (best predictor of long-term response)

Plasma HCV levels may, for example, be determined using Roche Diagnostics Taqman assay or the RealTime HCV Assay (Abbott).

The subject may be infected with HCV of a genotype selected from the group consisting of 1a, 1b, 2, 3, 4, 5 or 6. In some embodiments the genotype of the HCV is 1a. In some embodiments the genotype of the HCV is 1b. In some embodiments the subject is treatment naive.

In some embodiments, if the viral load at about week 2, or at about week 4, or at about weeks 8 of combination treatment period is effectively zero (RNA-ve), the duration of the treatment period, such as the combined treatment may be about 8-about 24 weeks, such as about 12 weeks.

In some embodiments, the combination treatment of the present invention is capable of reducing the level of HCV infection (titre) by at least 2 fold, such as at least 3 fold, such as at least 4 fold. In some embodiments the combination treatment provides a sustained viral response (SVR) in the subject. In some embodiments the combination treatment may provide a cure.

The activity of inhibitors of HCV activity may be measured by any of the suitable methods known to those skilled in the art, including in vivo and in vitro assays. For example, the HCV NS5B inhibitory activity of the compounds of formula I can determined using standard assay procedures described in Behrens et al, EMBO J. 1996 15:12-22, Lohmann et al, Virology 1998 249:108-118 and Ranjith-Kumar et al, J. Virology 2001 75:8615-8623.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A daily/weekly/monthly dosage may, for example be, between about 0.1 and about 500 mg/kg body weight, such as between 0.1 and about 100 mg/kg body weight such as between 0.1 and 1 mg/kg body weight per day, or between 1.0 and about 10 mg/kg body weight per day. Thus, for administration to a 70 kg person, in some embodiments, the dosage range may be about 7 mg to 0.7 g per day. The daily dosage can be administered as a single dosage or in divided dosages, typically between 1 and 5 dosages per day. Generally, in some embodiments, treatment is initiated with smaller dosages which are less than the optimum dose of the compound. Thereafter, the dosage may be increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

A therapeutically effective amount of a compound of the present invention, and optionally one or more additional antiviral agents, is an amount effective to reduce the viral load or achieve a sustained viral response to therapy. Useful indicators for a sustained response, in addition to the viral load include, but are not limited to liver fibrosis, elevation in serum transaminase levels and necro inflammatory activity in the liver. One common example, which is intended to be exemplary and not limiting, of a marker is serum alanine transminase (ALT) which is measured by standard clinical assays. In some embodiments of the invention an effective treatment regimen is one which reduces ALT levels to less than about 45 IU/mL serum.

The term “sustained viral response” (SVR; also referred to as a “sustained response” or a “durable response”), as used herein, refers to the response of an individual to a treatment regimen for HCV infection, in terms of serum HCV titer. For example, a “sustained viral response” may refer to no detectable HCV RNA (e.g., less than about 500, less than about 200, or less than about 100 genome copies per milliliter serum) found in the patient's serum for a period of at least about one month, at least about two months, at least about three months, at least about four months, at least about five months, and/or at least about six months following cessation of treatment. When using the Abbot HCV detection kit, for example, SVR is considered to be <12 IU/ml. SVR24 or SVR48 is typically used. SVR24 refers to no detectable HCV RNA at week 24 following treatment cessation. SVR48 refers to no detectable HCV RNA at week 48 following treatment cessation. The SVR rate refers to the proportion of subjects who, on a particular treatment regimen, illustrate a SVR. SVR24 refers to no detectable HCV RNA at week 24 following treatment cessation. Genotypes 1 and 4 usually require longer treatment durations to achieve SVR, and typically only 40 to 50% of patients infected with genotype 1 HCV show SVR on peg-interferon/RBV treatment. SVR rates in black and in HIV infected patients are typically only 20-30%. The combination treatment of the present invention may decrease the period required to achieve SVR. The combination treatment of the present invention may increase the SVR rate.

Resistance to compound 2. The development of resistance to direct acting agents, such as HCV protease inhibitors is a major concern for the employment of these compounds in the clinic. There is therefore a need to provide treatments for HCV which avoid the development of or reduce the prevalence of the development of resistance to direct acting agents. It is an aim of the present invention that this may be achieved by the use of combinations of direct acting agents, such as HCV protease inhibitors with inhibitors of miR-122.

### Non-Limited Examples of a Clinical Protocol

Drug/drug interaction study: This is an open-label drug interaction study to assess the safety, tolerability, and pharmacokinetics of miravirsen and compound 2, e.g. when co-administered in healthy subjects: Approximately 5 subjects are administered a single dose of compound 2 on Day 1 and have 24-hour serial blood collection to assess the single-dose pharmacokinetic profile of telaprevir. Subjects receive TID doses of compound 2 for 5 days (Days 2-6). On Day 7, subjects receive another single-dose of compound 2 and have 24-hour serial pharmacokinetic blood sample collection. Subjects receive single doses of miravirsen (7 mg/kg) on Days 8, 15, 22, 29 and 36. On Day 15, subjects have 24-hour serial blood and urine collection to assess the single-dose pharmacokinetic profile of miravirsen. A single pharmacokinetic blood sample is collected on Days 22 and 29 just prior to the third and fourth dose of miravirsen (to determine trough concentration). On Day 30, a single dose of compound 2 is administered and subjects will have 24-hour serial pharmacokinetic blood sample collection. Subjects receive TID doses of compound 2 for 5 days (Days 31-35). On Day 36, subjects receive another single dose of compound 2 and a single dose of miravirsen followed by 24-hour serial blood collection to assess the pharmacokinetic profile of both compound 2 and miravirsen. Additionally, urine is collected for a 24-hour period to assess the pharmacokinetic profile of miravirsen. Subjects return for weekly follow-up visits on Days 43, 50, 57, 64 and 71 with an end of study visit on Day 78 for total study participation duration of 12 weeks (excluding screening). Safety and tolerability will be assessed by evaluation of adverse events, physical examinations, vital signs, routine clinical safety laboratory assessments and electrocardiograms.

In clinical trials between multiple different DAA, drug-drug interactions may result in adverse events. Miravirsen has shown remarkable knock down of HCV in monotherapy clinical trials (see e.g. Example 6). Furthermore miravirsen is not metabolized by cytochrome 450 mechanisms, and is considered a particularly suited non direct acting agent for use in HCV combination treatment as described herein. In this respect, the therapeutic profile makes it an ideal replacement for interferon based treatments, either in combination with compound 2, and/or compound 3. Indeed, our results indicate miravirsen has an excellent toxicity profile with a therapeutic index at least an order of magnitude greater than interferon alpha-2b. In a four week clinical trial to assess the safety and tolerability, treatment with miravirsen was effective in reducing the HCV titre below detection. Miravirsen has also proven effective against all HCV genotypes in vitro. In some embodiments, the combination treatment is in the absence of a cytochome P450 inhibitor, such as ritonavir.

Compound 1 (miravirsen) and compound 2 (e.g. telaprevir or boceprevir or others listed herein) optionally with ribavirin may prevent virological breakthrough within the first 12 weeks of combination therapy (MIR+DAA), and provide SVR at 12, 24 & 48 weeks after the last dose of combination therapy. miravirsen is dosed for 4 weeks as monotherapy (e.g. four or five doses over 29 days), and then continue for a further (e.g.) 12 weeks in combination with compound 2 with (Cohort 1) and without (Cohort 2) ribavirin. 20 chronic HCV, genotype 1 treatment naïve subjects with prior to treatment plasma HCV RNA>75,000 IU/mL HCV viral load is determined at weeks 8(RVR), 28(SVR12) and 40 (SVR24) and SVR48. Samples are collected for resistance analysis and viral breakthrough/relapse. The pre-treatment dosing of compound 1 (e.g. miravirsen) may be for example 7 mgs/kg×4 weekly doses (suitably days 1, 8, 15, 22). Other pre-treatment dose regimes are described herein (e.g. one or 2 (pre-) doses at, for example up to 12 mgs/kg). Combination period dosing may be, for example, an initial dose of 7 mg/kg followed by subsequent dosing at 5 mgs/kg starting on week 5/Day 29 followed by 5 mg/kg every two weeks until week 16 (day 112). Ribavirin may be dosed at 100 mg/day<75 kgs or 1200 mg/day>75 kgs. Compound 2 (e.g. telaprevir) may be administered, for example 750 mg three times daily during the combination treatment period, optionally used in combination with ritonavir. When ABT-450 is used as compound 2 it may be used with ritonavir (ABT-450/r).

Examples of Ongoing or Planned Clinical Trials with Combination Treatments:
Telaprevir (TVR) (PI)+VX-222(NNI)+/−RBV (e.g. GT1 treatment naïve)
GS-9256 (PI)+tegobuvir (NNI)+/−RBV (e.g. GT1 treatment naïve)
GS-9256 (PI)+tegobuvir (NNI)+GS-5885 (NSSA)+RBV (e.g. GT1 treatment naïve)
Danoprevir (PI)+mercitabine (NUC)+/−RBV (e.g. GT1 treatment naïve)
ABT-450/r (PI)+ABT-072 (NNI)+RBV (e.g. GT1 treatment naïve)
ABT-450/r (PI)+ABT-333 (NNI)−RBV (e.g. GT1 treatment naïve)
TMC435 (PI)+PSI-7977 (NUC)+/−RBV (e.g. GT1 treatment naïve)
BMS-790052 (PI)+BMS790052 (NS5a)+/−pegIFN/RBV (e.g. GT1 treatment Experienced)
ABT-450/r+ABT-333 (NNI)+RBV (e.g. GT1 treatment Experienced)
The above combinations may represent compound 2 and compound 5 (or multiple compound 5s) and optionally compound 3 in the present invention.

In some embodiments, compound 5 may be a NS5B polymerase inhibitor, e.g. selected from the group consisting of:

Nucleos(t)ide NS5B polymerase inhibitors

| | |
|---|---|
| Valopicitabine (NM-283) | Idenix/Novartis |
| RG7128 | Roche/Pharmasset |
| IDX184 | Idenix |
| R1626 | Roche |
| PSI-7977 | Pharmasset |
| PSI-938 | Pharmasset |
| INX-189 | Inhibitex |

Non-nucleoside NS5B polymerase inhibitors

| | |
|---|---|
| BILB 1941 | Boehringer Ingelheim |
| BI 207127 | Boehringer Ingelheim |
| MK-3281 | Merck |
| Filibuvir (PF-00868554) | Pfizer |
| VX-916 | Vertex |
| VX-222 | Vertex |
| VX-759 | Vertex |
| ANA598 | Anadys |
| ABT-333 | Abbott |
| ABT-072 | Abbott |
| Nesbuvir (HCV-796) | ViroPharma/Wyeth |
| Tegobuvir (GS-9190) | Gilead |
| IDX375 | Idenix |

PSI-7977, also known as GI-7977 after the acquisition of Pharmasset by Gilead, is a prodrug that is metabolized to the active antiviral agent 2′-deoxy-2′-α-fluoro-β-C-methyluridine-5′-monophosphate

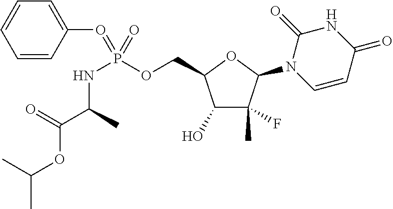

Typical dose is e.g. 400 mgs

### VX-222:

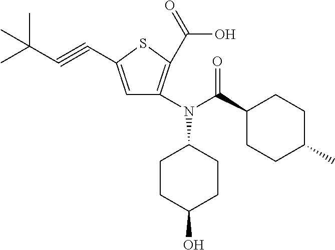 [SMILES: CC(C)(C)C#Cc1cc(N(C(=O)[C@H]2CC[C@H](C)CC2)[C@H]2CC[C@H](O)CC2)c(C(=O)O)s1]

In some embodiments, compound 5 may be a NS5A protein inhibitor, e.g. selected from the group consisting of:

| Drug name | |
|---|---|
| NS5A Protein inhibitors | Company |
| ACH-2928 | Achillion |
| ACH-3102 | Achillion |
| AZD7295 | AstraZeneca (Formerly Arrow) |
| EDP-239 | Enanta |
| MK4882 | Merck |
| IDX719 | Idenix |
| BMS-790052 | BMS |
| GS 5885 | Gilead |
| GSK233680 | GSK |

In some embodiments, compound 5 is BMS-790052—also known as daclatasvir (from Bristol-Myers Squibb). BMS 790052 may also be known as EBP 883, or Carbamic acid, N,N′-[[1,1′-biphenyl]-4,4′-diylbis[1H-imidazole-5,2-diyl-(2S)-2,1-pyrrolidinediyl[(1S)-1-(1-methylethyl)-2-oxo-2,1-ethanediyl]]]bis-, C,C′-dimethyl ester. BMS-790052 is also available from ApisChemical, and is described as Dimethyl(2S,2′S)-1,1′-((2S,2′S)-2,2′-(4,4′-(biphenyl-4,4′-diyl)bis(1H-imidazole-4,2-diyl))bis(pyrrolidine-2,1-diyl))bis(3-methyl-1-oxobutane-2,1-diyl)dicarbamate, with CAS Reg No: 1009119-64-5.

Compound 1: miR-122 Inhibitor

As reported in Young et al., JACS 2010, 132, 7976-7981) (hereby incorporated by reference), it is possible to assay for small molecule inhibitors of miR122 and small molecule inhibitors of miR-122 are known, such as those illustrated below:

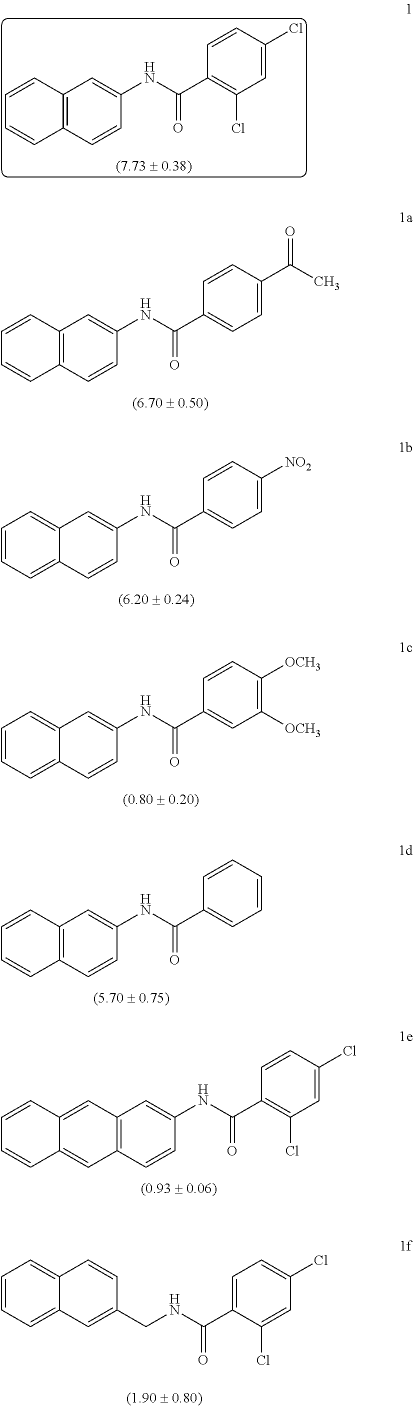

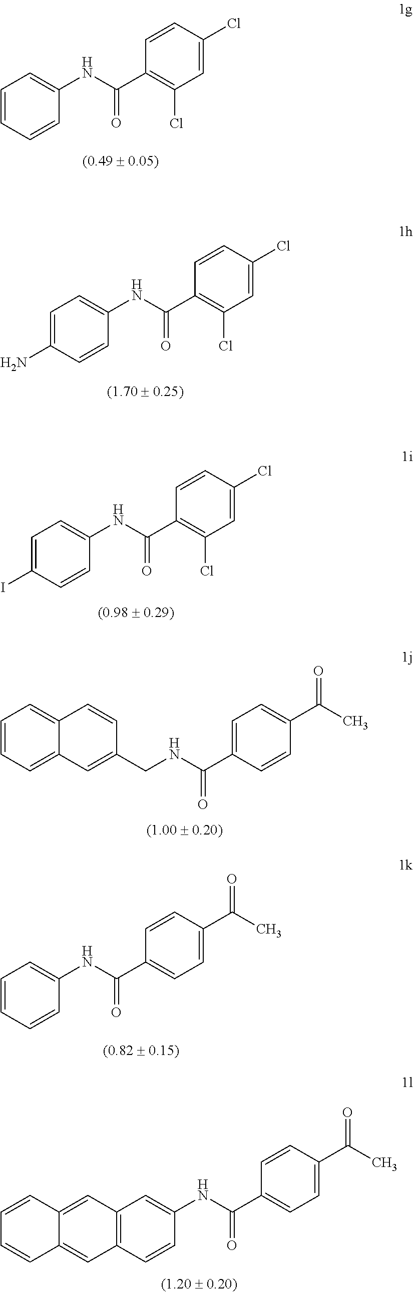 [SMILES: O=C(Nc1ccccc1)c1ccc(Cl)cc1Cl ; Nc1ccc(NC(=O)c2ccc(Cl)cc2Cl)cc1 ; O=C(Nc1ccc(I)cc1)c1ccc(Cl)cc1Cl ; CC(=O)c1ccc(C(=O)NCc2ccc3ccccc3c2)cc1 ; CC(=O)c1ccc(C(=O)Nc2ccccc2)cc1 ; CC(=O)c1ccc(C(=O)Nc2ccc3cc4ccccc4cc3c2)cc1]

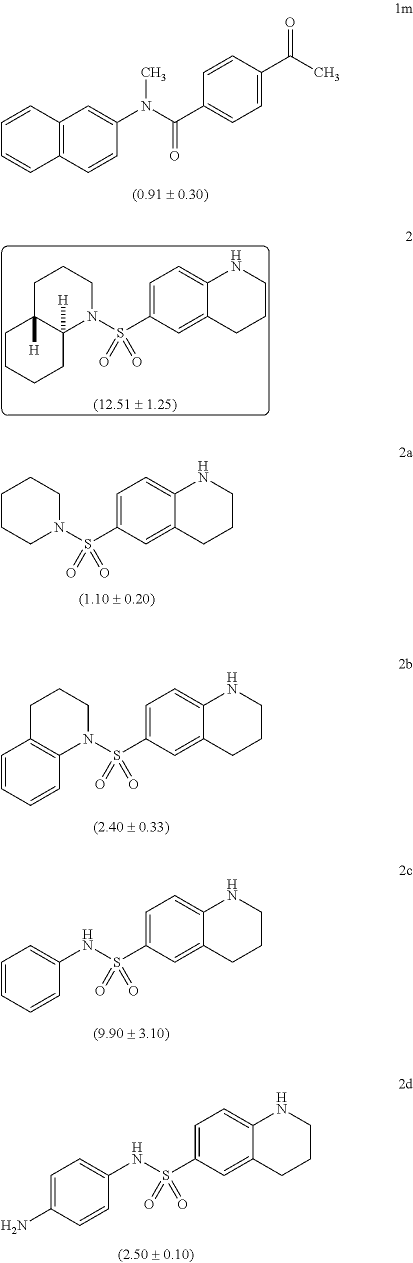

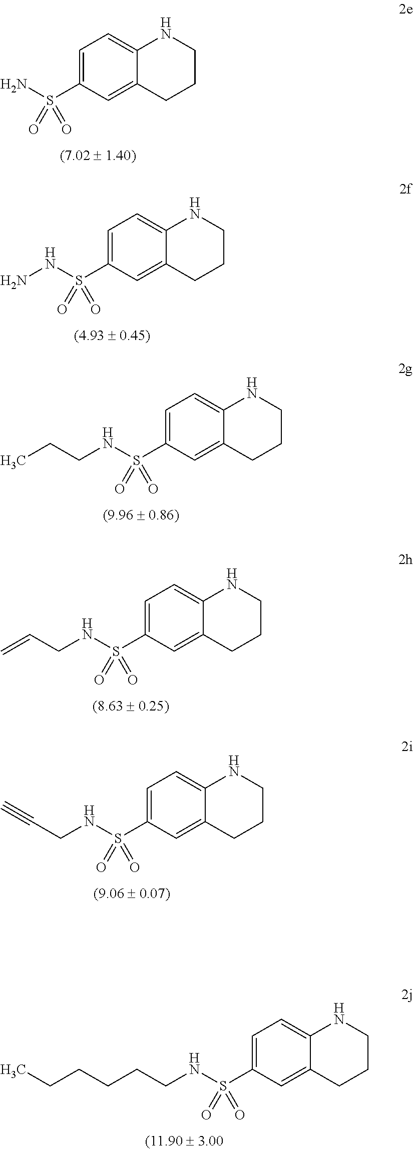 [SMILES: NS(=O)(=O)c1ccc2c(c1)CCCN2 ; NNS(=O)(=O)c1ccc2c(c1)CCCN2 ; CCCNS(=O)(=O)c1ccc2c(c1)CCCN2 ; C=CCNS(=O)(=O)c1ccc2c(c1)CCCN2 ; C#CCNS(=O)(=O)c1ccc2c(c1)CCCN2 ; CCCCCCNS(=O)(=O)c1ccc2c(c1)CCCN2]

The numerical values refer to luciferase expression due to miR-122 deprepression, and values greater than 1 indicate miR-122 inhibition.

### Compound 1: Antisense Oligomer

The miR-122 inhibitor may be an antisense oligomer

In some embodiments, the antimiR-122 compound is an antisense oligomer targeting microRNA-122. The sequence of miR-122 is well conserved between different mamalian species (mirbase, Sanger Center, UK).

>hsa-mir-122 precursor sequence (miRBase) MI0000442:

(SEQ ID NO 4)
CCUUAGCAGAGCUGUGGAGUGUGACAAUGGUGUUUGUGUCUAAACU
AUCAAACGCCAUUAUCACACUAAAUAGCUACUGCUAGGC

The mature hsa-miR-122 sequence (miRBase) MIMAT0000421:

(SEQ ID NO 1)
UGGAGUGUGACAAUGGUGUUUG

MicroRNA-122 antagonists of the invention are compounds, including but not limited to antisense oligomers targeting miR-122 (e.g. SEQ ID NO 1 or SEQ ID NO 4). The antisense oligomer may comprise at least 6, consecutive nucleobases which are complementary to a part of a miR-122 sequence, such as the mature hsa-miR-122 sequence.

In some embodiments, the antimiR-122 oligonucleotide is designed as a mixmer that is essentially incapable of recruiting RNAseH. Oligonucleotides that are essentially incapable of recruiting RNAseH are well known in the literature, in example see WO2007/112754, WO2007/112753, or WO2009/043353. Mixmers may be designed to comprise a mixture of affinity enhancing nucleotide analogues, such as in non-limiting example 2′-O-alkyl-RNA monomers, 2′-amino-DNA monomers, 2′-fluoro-DNA monomers, LNA monomers, arabino nucleic acid (ANA) mononmers, 2′-fluoro-ANA monomers, HNA monomers, 3 fluoro hexitol monomers (3F HNA), INA monomers, 2′-MOE-RNA (2′-O-methoxyethyl-RNA), 2′Fluoro-DNA, and LNA. In a further embodiment, the oligonucleotide does not include any DNA or RNA nucleotides, but is solely composed of affinity enhancing nucleotide analogues, such a molecule is may also be termed a totalmer. In some embodiments, the mixmer only comprise one type of affinity enhancing nucleotide analogues together with DNA and/or RNA. In some embodiments, the oligonucleotide is composed solely of one or more types of nucleotide analogues, such as in non-limiting example 2′-O-alkyl-RNA monomers, 2′-amino-DNA monomers, 2′-fluoro-DNA monomers, LNA monomers, arabino nucleic acid (ANA) mononmers, 2′-fluoro-ANA monomers, HNA monomers, INA monomers, 2′-MOE-RNA (2′-O-methoxyethyl-RNA), 2′Fluoro-DNA, and LNA.

### Length

In some embodiments the antisense oligonucleotide has a length of 7-25 (contiguous) nucleotides, such as 8, 9, 10, 11, 12, 13, 14, 15, 16, 17, 18, 19, 20, 21, 22, 23, or 24 (contiguous) nucleotides. In some embodiments, the antisense oligonucleotide has a length of 7-10 (contiguous) nucleotide, or in some instances 7-16 nucleotides. In some embodiments, the antisense oligonucleotide at least 8 (contiguous) nucleotides in length, between 10-17 or 10-16 or 10-15 (contiguous) nucleotides, such as between 12-15 (contiguous) nucleotides.

Oligomers which are Essentially Incapable of Recruiting RNAseH

EP 1 222 309 provides in vitro methods for determining RNaseH activity, which may be used to determine the ability to recruit RNaseH. A oligomer is deemed capable of recruiting RNase H if, when provided with the complementary RNA target, it has an initial rate, as measured in pmol/l/min, of at least 1%, such as at least 5%, such as at least 10% or less than 20% of the equivalent DNA only oligonucleotide, with no 2′ substitutions, with phosphorothioate linkage groups between all nucleotides in the oligonucleotide, using the methodology provided by Example 91-95 of EP 1 222 309.

In some embodiments, an oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of EP 1 222 309.

It should be recognised that oligonucleotides which are mixmers or totalmers are usually essentially incapable of recruiting RNAseH and as such where we use the term essentially incapable or recruiting RNaseH herein, in some embodiments, such a term may be replaced with the term mixmer or totalmer, as defined herein, even if, in some instances such oligomers actually do possess significant ability to recruit RNaseH, such as when using DNA mixmers with alpha-L-oxy-LNA.

Examples of Modulators of microRNA-122 Useful in the Invention

Specially preferred compounds for use in the present invention are those that target microRNA-122—as such oligomers which are capable of inhibiting microRNA-122 in a cell, such as in a subject infected with HCV. The sequence of miR-122 can be found in the microRNA database “mirbase” (http://microma.sangerac.uk/sequences/). Inhibitors of microRNA-122 have been described in numerous patents and articles and are well known to the person skilled in the art. In some embodiments, examples of such documents describing useful microRNA-122 modulators are WO2007/112754, WO2007/112753, or WO2009/043353 all of which are hereby incorporated by reference. In some embodiments, such microRNA-122 modulators are those described in WO2009/20771, WO2008/91703, WO2008/046911, WO2008/074328, WO2007/90073, WO2007/27775, WO2007/27894, WO2007/21896, WO2006/93526, WO2006/112872, WO2005/23986, or WO2005/13901, all of which are hereby incorporated by reference.

In some embodiments the micro-RNA-122 antagonist is an oligomer which is complementary to miR-122 or a (corresponding) contiguous nucleobase sequence thereof. As such, oligomers which are complementary to miR-122 comprise or consist of a sequence of at least seven contiguous nucleotides which are complementary to a part of, or the entire length of the human miR-122 sequence. In this context a part of is at least 6, such as at least 7 or at least 8 contiguous nucleotides which are 100% complementary to a sequence found within micro-RNA 122 sequence, such as the mature has-miR-122 sequence. In certain embodiments, oligomers which are complementary to miR-122 comprise or consist of a contiguous nucleotide sequence which is complementary to the has-miR-122 seed sequence, i.e. comprises or consists of the contiguous nucleotide sequence 5′-CACTCC-3′—referred to herein as the “seed match region”.

In some embodiments, the sequence 5′-CACTCC-3′ is positioned at positions 1-6, 2-7 or 3-8 of the oligomer, counting from the 3′ end. In some embodiments, the sequence 5′-CACTCCA-3′ is positioned at positions 1-7 or 2-8 of the oligomer, counting in from the 3′-end. An oligomer which consists of a contiguous nucleotide sequence may further comprise non-nucleotide components, such as a 5′ or 3′ non nucleotide conjugation group.

In some embodiments, the oligomer consists of or comprises just the contiguous nucleotide sequence, without, e.g., conjugation groups. Oligomers which are complementary to miR-122 may comprise or consist of a contiguous sequence of 7, 8, 9, 10, 11, 12, 13, 14, 15, 16, 17, 18, 19, 20, 21 or 22 nucleotides which are complementary to a part, or the entire, has-miR-122 sequence. Oligomers which are complementary to only a part of the has-miR-122 sequence may be less than 22 nucleotides long, and may consist of or comprise a contiguous nucleotide sequence which consists of the complement of a part of the miR-has-miR122 sequence (i.e. a contiguous nucleotide sequence which is complementary to a corresponding sub-region of has-miR-122).

In some embodiments the oligomer may comprise or consist of 2′ substituted nucleosides, such as 2′MOE, 2′OMe and/or 2′fluro. By way of example, Davis et al, NAR 2008 Vol 37, No 1, discloses 2′-fluoro/2′-methoxyethyl (2′MOE) modified antisense oligonucleotide (ASO) motif with dramatically improved in vivo potency. Davis et al., is hereby incorporated by reference. The 2′MOE/2′ fluoro oligo (e.g. mixmer) may, in some embodiments be 12, 13, 14, 15, 17, 18, 19, 20, 21 or 22 nucleotides in length. See U.S. provisional application 61/566,027, filed 2 Dec. 2011 for further 2′MOE/2′ fluoro oligo designs, (page 24, line 4—page 26 line 13, which are hereby incorporated by reference.

Other oligomer compounds which may be used as compound 1 include, but are not limited to, those oligonucletides as disclosed in table 1 of PCT/DK2008/000344, which discloses antimiRs targeting the microRNAs as published in miRbase and which is specifically incorporated by reference to provide oligomers which may be used in the methods of the present invention. Equivalent antimiRs can be designed by matching the −2 to −8/-9 or −10 positions (for 7, 8 or 9 mers) of mature microRNA-122 (counting from the terminal 5′ nucleotide of the microRNA (i.e. at the −1 position).

| | | SEQ | | SEQ | | SEQ |
|---|---|---|---|---|---|---|
| microRNA | 9-mers | ID | 8-mers | ID | 7-mers | ID |
| hsa-miR-122 | TCACACTCC | 18 | CACACTCC | 68 | ACACTCC | 118 |

Further LNA Compounds Targeting microRNA-122. The following specific compounds, as disclosed in PCT/DK2008/000344, which may be used in the methods of the present invention.

| SEQ | | |
|---|---|---|
| ID | Compound | Target |
| NO | Sequence | microRNA |
| 2 | CcAttGTcaCaCtCC | miR-122 |
| N/A | CACACTCC | miR-122 |

Further specific compounds targeting miR-122, which may be used are those disclosed in Table 1 of WO2007/112754 and WO2007/112753 and are hereby incorporated by reference. Further specific compounds targeting miR-122, which may be used are those disclosed in Table 4 of U.S. provisional application 61/566,027, filed 2 Dec. 2011 and are hereby incorporated by reference.

### Miravirsen (SPC3649)

In a preferred embodiment the antisense oligomer is miravirsen (SPC3649) which has the formula: 5′-^{m}Cₛ^{o}cₛAₛ^{o}tₛtₛGₛ^{o}Tₛ^{o}cₛaₛ^{m}Cₛ^{o}aₛ^{m}Cₛ^{o}tₛ^{m}Cₛ^{om}C^{o}-3′

wherein; a lowercase letter identifies a DNA unit, and an upper case letter identifies a LNA unit, ^{m}C identifies a 5-methylcytosine LNA, subscript ₛ identifies a phosphorothioate internucleoside linkage, and wherein LNA units are beta-D-oxy, as identified by a ^{o} superscript after LNA residue.

### Compound 2: HCV NS3/4a Protease Inhibitors

There are numerous HCV NS3/4A protease inhibitors which have obtained market approval or are presently in clinical trials, such as those disclosed in the following table:

| Pharma | NS3A protease (PI) | References (hereby incorporated by reference) and exemplorary structures. |
|---|---|---|
| Abbott | ABT-450 (often used | |
| | with Ritonavir) | |
| Boehringer- Ingelheim | BI 201335 | 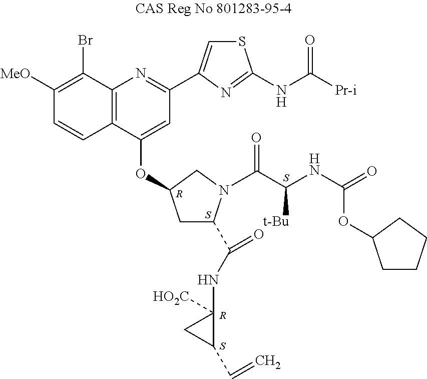 |
| BMS | BMS-650032 (Asunaprevir) | 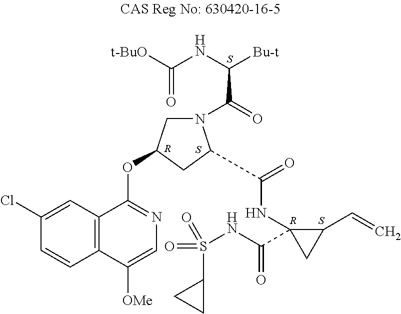 |
| Gilead | GS-9256 | See WO 2011054834 |
| Idenix | IDX320 | See WO 2011009961 |
| Intermune | ITMN 191 (Danoprevir) | 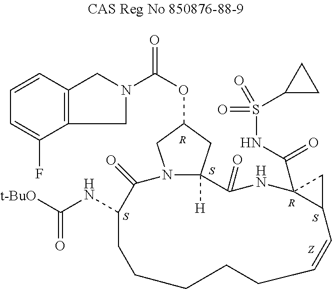 |
| Merck/Scheering | SCH900518 (Narlaprevir), MK-5172 | 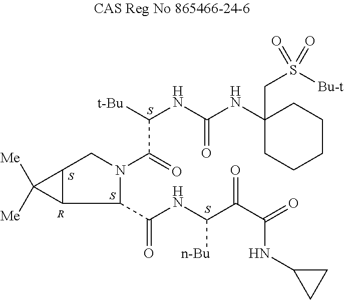 |
| | MK-5172 | 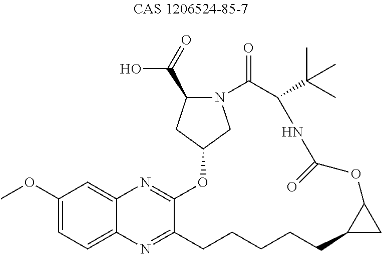 [SMILES: COc1ccc2nc3c(nc2c1)O[C@@H]1C[C@@H](C(=O)O)N(C1)C(=O)[C@H](C(C)(C)C)NC(=O)COC1C[C@H]1CCCCC3] |
| Roche/Genentech | ITMN- | See above |
| | 191 = RG7227 = danoprevir | |
| Tibotec/J&J/Medivir | TMC 435340 | See W02011/054834 and W02011/060408, |
| Vertex | Telaprevir, VX-500, | See below |
| | VX-985 and VX-813 | |
| | SZ:Vaniprevir (MK- 7009) ACH-1625 | 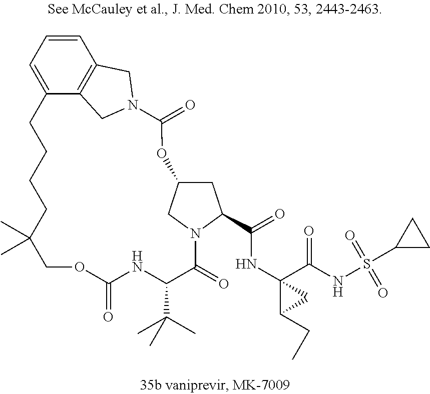 [SMILES: CC[C@@H]1C[C@]1(NC(=O)[C@@H]1C[C@@H]2CN1C(=O)[C@H](C(C)(C)C)NC(=O)OCC(C)(C)CCCCc1cccc3c1CN(C3)C(=O)O2)C(=O)NS(=O)(=O)C1CC1] |

Inhibitors of the HCV NS3/4A protease also have been identified as potentially useful for treatment of HCV. Protease inhibitors in clinical trials or which have market authorisation include VX-950 (Telaprevir, Vertex), SCH503034 (Broceprevir, Schering), TMC435350 (Tibotec/Medivir) and ITMN-191 (Intermune). Other protease inhibitors in earlier stages of development include MK7009 (Merck), VBY-376 (Virobay), ACH2684 (Achillion), AVL-181 (Avila), IDXSCA/IDXSCB (Idenix), BI12202 (Boehringer), VX-500 (Vertex), PHXI 766 Phenomix).

### Telaprevir

According to http://en.wikipedia.orq/wiki/File:Telaprevir.svq, Teaprevir has the structure

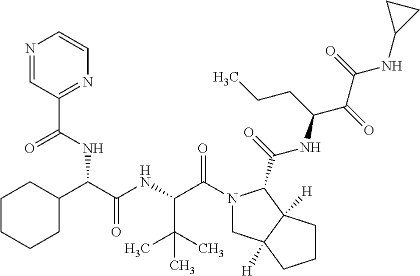 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

Systematic IUPAC Name: (1S,3aR,6aS)-2-[(2S)-2-[[(2S)-2-Cyclohexyl-2-(pyrazine-2-carbonylamino)acetyl]amino]-3,3-dimethylbutanoyl]-N-[(3S)-1-(cyclopropylamino)-1,2-dioxohexan-3-yl]-3,3a,4,5,6,6a-hexahydro-1H-cyclopenta[c]pyrrole-1-carboxamide

Telaprevir may be administered in a unit dose of, for example between about 250 and about 1000 mg, such as about 750 mg/kg. Typically once, twice, three or four times daily, such as three times daily for the duration of the pre-treatment period and/or combination treatment period.

### Boceprevir

According to http://en.wikipedia.org/wiki/File:Boceprevir.svq, Boceprevir has the structure:

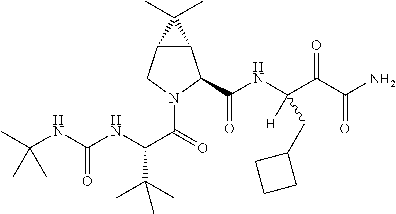 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

Systematic IUPAC Name: (1R,2S,5S)—N-[(2 [symbol] )-4-amino-1-cyclobutyl-3,4-dioxobutan-2-yl)]-3-{(2S)-2-[(tert-butylcarbamoyl)amino]-3,3-dimethylbutanoyl}-6,6-dimethyl-3-azabicyclo[3.1.0]hexane-2-carboxamide

Boceprevir may be administered in a unit dose of, for example between about 250 and about 1000 mg, such as about 800 mg/kg. Typically once, twice, three or four times daily, such as three times daily for the duration of the pre-treatment period and/or combination treatment period.

### Compounds 3 and 4: Standard of Care

Prior to February 2011, the standard of care of HCV infection was a combination of compound 4 (interferon) with compound 3 (ribavirin). As of June 2012, SOC is still dependant on IFN/RBV combination, which are used in conjunction with a NS3/4A protease inhibitor, telaprevir or boceprevir.

A typical previous standard of care regimen may be treatment of peginterferon alfa-2b (1.5 micrograms/kg once weekly) plus ribavirin (800 to 1400 mg daily based on patient weight), for a period of 48 weeks.

Examples of the interferons include, but are not limited to pegylated rIFN-alpha 2b, pegylated rIFN-alpha 2a, rIFN-alpha 2b, rIFN-alpha 2a, consensus IFN alpha (infergen), feron, reaferon, intermax alpha, r-IFN-beta, infergen and actimmune, IFN-omega with DUROS, albuferon, locteron, Albuferon, Rebif, oral interferon alpha, IFNalpha-2b XL, AVI-005, PEG-Infergen, and pegylated IFN-beta.

Ribavirin analogs and the ribavirin prodrug viramidine (taribavirin) have been administered with interferons to control HCV. In some embodiments, compound 3 may therefore also comprises antiviral ribavirin analogs and antiviral ribavirin derivatives, as well as ribavirin pro-drugs.

Exemplary treatment options for hepatitis C (HCV) include interferons, e.g., interferon alfa-2b, interferon alfa-2a, and interferon alfacon-1. Less frequent interferon dosing can be achieved using pegylated interferon (interferon attached to a polyethylene glycol moiety which significantly improves its pharmacokinetic profile). Combination therapy with interferon alfa-2b (pegylated and unpegylated) and ribavarin has also been shown to be efficacious for some patient populations. In some embodiments, the interferon is pegylated interferon-alpha.

In some embodiments, the interferon is Pegylated interferon alfa-2a. A suitable Pegylated interferon alfa-2a is Pegasys, (pegylated with a branched 40 kDa PEG chain) an antiviral drug discovered at the pharmaceutical company F. Hoffmann-La Roche; it has a dual mode of action—both antiviral and on the immune system. The addition of polyethylene glycol to the interferon, through a process known as pegylation, enhances the half-life of the interferon when compared to its native form. This drug is approved around the world for the treatment of chronic hepatitis C (including patients with HIV co-infection, cirrhosis, ‘normal’ levels of ALT). Peginterferon alfa-2a is a long acting interferon.

In some embodiments, the HCV infected subjects are further treated with an effective amount of ribavirin or an antiviral derivative thereof.

### Compound 3—Ribavirin

According to http://en.wikipedia.org/wiki/File:Ribavirin.svg, the structure of ribavirin is

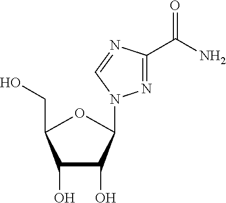

And the systematic IUPAC names is 1-[(2R,3R,4S,5R)-3,4-dihydroxy-5-(hydroxymethyl)oxolan-2-yl]-1H-1,2,4-triazole-3-carboxamide.

In Europe and the U.S. the oral (capsule or tablet) form of ribavirin is used in the treatment of hepatitis C, in combination with pegylated interferon drugs.[1]

Ribavirin (brand names: Copegus, Rebetol, Ribasphere, Vilona and Virazole) is an anti-viral drug indicated for severe RSV infection (individually), hepatitis C infection (used in conjunction with peginterferon alfa-2b or peginterferon alfa-2a) and other viral infections. Ribavirin is a prodrug, which when metabolised resembles purine RNA nucleotides. In this form it interferes with RNA metabolism required for viral replication. How it exactly affects viral replication is unknown; many mechanisms have been proposed for this (see Mechanisms of Action, below) but none of these has been proven to date. Multiple mechanisms may be responsible for its actions.

The primary observed serious adverse side effect of ribavirin is hemolytic anemia, which may worsen preexisting cardiac disease. The mechanism for this effect is due to ribavarin's buildup inside erythrocytes. Oxidative damage to erythrocyte cell membrane is usually inhibited by glutathione; however, with reduced ATP levels caused by ribavirin, glutathione levels are impaired, permitting oxidative erythrocyte cell lysis. The gradual loss of erythrocytes leads to anemia. The anemia is dose-dependent and may sometimes be compensated by decreasing dose. Ribavirin is also a teratogen in some animals species and thus poses a theoretical reproductive risk in humans, remaining a hazard as long as the drug is present, which can be as long as 6 months after a course of the drug has ended.

In some embodiments, an antiviral ribavirin derivative may be employed in place of the ribavirin: Ribavirin is possibly best viewed as a ribosyl purine analogue with an incomplete purine 6-membered ring. This structural resemblance historically prompted replacement of the 2′ nitrogen of the triazole with a carbon (which becomes the 5′ carbon in an imidazole), in an attempt to partly “fill out” the second ring—but to no great effect. Such 5′ imidazole riboside derivatives show antiviral activity with 5′ hydrogen or halide, but the larger the substituent, the smaller the activity, and all proved less active than ribavirin.[13] Note that two natural products were already known with this imidazole riboside structure: substitution at the 5′ carbon with OH results in pyrazomycin/pyrazofurin, an antibiotic with antiviral properties but unacceptable toxicity, and replacement with an amino group results in the natural purine synthetic precursor 5-aminoimidazole-4-carboxamide-1-β-D-ribofuranoside (AICAR), which has only modest antiviral properties. Derivatization of the triazole 5′ carbon, or replacement of it with a nitrogen (i.e., the 1,2,4,5 tetrazole 3-carboxamide) also results in substantial loss of activity, as does alkyl derivatization of the 3′ carboxamide nitrogen. The 2′ deoxyribose version of ribavirin (the DNA nucleoside analogue) is not active as an antiviral, suggesting strongly that ribavirin requires RNA-dependent enzymes for its antiviral activity. Antiviral activity is retained for acetate and phosphate derivation of the ribose hydroxyls, including the triphosphate and 3′,5′ cyclic phosphates, but these compounds are no more active than the parent molecule, reflecting the high efficiency of esterase and kinase activity in the body. Taribavirin (viramidine) is the most successful ribavirin derivative to date is the 3-carboxamidine derivative of the parent 3-carboxamide, and now called taribavirin (former names viramidine and ribamidine). This drug shows a similar spectrum of antiviral activity to ribavirin, which is not surprising as it is now known to be a pro-drug for ribavirin. Viramidine, however, has useful properties of less erythrocyte-trapping and better liver-targeting than ribavirin. The first property is due to viramidine's basic amidine group which inhibits drug entry into RBCs, and the second property is probably due to increased concentration of the enzymes which convert amidine to amide, in liver tissue. Viramidine is in phase III human trials and may one day be used in place of ribavirin, at least against certain kinds of viral hepatitis. Viramidine's slightly superior toxicological properties may eventually cause it to replace ribavirin in all uses of ribavirin.

### Dosing the Antisense Oligomer

The oligomer may, for example, be administered parentally. For parenteral, subcutaneous, intradermal or topical administration the formulation may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline. Other methods of administration may be used, for example oral, nasal, rectal administration.

The administration regimens for compound 1 below may refer to the pre-treatment and/or the combination treatment periods:

Typically at least two successive administrations of compound 1 are administered to the subject in need of treatment. In some embodiments, the dosage interval between the at least two successive administrations is at least 2 weeks and optionally is no greater than 20 weeks. In some embodiments, the composition is in a unit dose form, such as each unit dose forming the whole or part of a single administration to the subject. The number of administrations may be more than 2, such as 3, 4, 5, 6, 7, 8, 9, 10, 11, 12, 13, 14, 15, 16 or more treatments. In some embodiments, the time interval between each administration of compound 1 is at least 14 days, such as at least 15, 16, 17, 18, 19, 20, 25, 30, 35, 40, 45, 50, 55, 60, 65, 70, 75, 80, 85, 90, 95, 100, 105, 110, 115, 120, or at least 125 days.

In some embodiments, the time interval between each administration of compound 1, such as miravirsen, may be one day, or more, such as 2 days, three days, four days, five days, six days, or weekly, or dosing every 8, 9, 10, 11, 12, 13 or biweekly. Each administration of compound 1 may be optimised as described herein to ensure an effective therapeutic amount is administered to the patient. In some embodiments each dose may be between about 0.1 mgs/kg and about 10 mgs/kg or about 12 mgs/kg, such as about 0.2 mgs/kg, such as about 0.3 mgs/kg, such as about 0.4 mgs/kg, such as about 0.5 mgs/kg, such as about 0.6 mgs/kg, such as about 0.7 mgs/kg, such as about 0.8 mgs/kg, such as about 0.9 mgs/kg, such as about 1 mg/kg, such as about 2 mgs/kg, such as about 3 mgs/kg, such as about 4 mgs/kg, such as about 5 mgs/kg, such as about 6 mgs/kg, such as about 7 mgs/kg, such as about 8 mgs/kg, such as about 9 mgs/kg, such as about 10 mgs/kg such as about 11 mgs/kg, such as about 12 mgs/kg.

The effectiveness of the dosages may for example be measured by the amount of viral genome (titre). In some embodiments, after the build up phase, a maintenance dosage could be given for a time period wherein the purpose is to maintain a relatively high activity or concentration of the compound in the target tissue, while e.g. the viral titre is decreased or other disease parameters are improved, after which the interval between each dosing could be increased or the dosage given at each dosing could be decreased or both, in order to maintain the disease at the new low level using the minimal needed effective dosage and at the same time obtain minimum side effects and the least inconvenience for the patient by having a high time interval in between administrations.

In some embodiments, after the build up phase, a maintenance dosage will be administered wherein the purpose is to maintain an effective concentration in the target tissue, in order to obtain the desired effect on important disease parameters, wherein the time interval in between each administration is large to avoid the inconvenience for the patient of the administration, and the dosage is kept to a minimum to avoid side effects while still maintaining the effect on the selected disease parameters.

In some embodiments, the time interval between the at least two dosages of compound 1, such as maintenance dosages, is selected from any one of at least 1, 2, 3, 4, 5, 6, 7, 8, 9, 10, 11, 12, 13, 14 days, or at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124 or at least 125 days. In some embodiments, the time interval between said at least two dosages, such as maintenance dosages, is selected from any one of at least 1 week, such as at least 2 weeks, such as at least 3, 4, 5, 6, 7, 8, 9, 10, 11, 12, 13, 14, 15, 16, 17 or at least 18 weeks. In some embodiments, the time interval between said at least two dosages, such as maintenance dosages, is selected from any one of at least % month, such as at least 1, 1½, 2, 2½, 3, 3½, 4 or at least 4½ month.

In some embodiments, the administration of compound 1 will be maintained for as long as the patient has symptoms of active disease, for example detectable HCV titre. In some embodiments, the treatment may be paused for a period, and subsequently resumed by an initial period of high or frequent dosing to re-build effective tissue concentrations of the compound, followed by maintenance treatment according to the description.

In some embodiments, the time interval between the at least two dosages of compound 1, such as the maintenance dosages, is at least 1 week or at least 14 days. In some embodiments, the time interval between dosages is at least 21 days. In some embodiments, the time interval between dosages is at least 4 weeks, or at least 1 month. In some embodiments, the time interval between dosages is at least 5 weeks. In some embodiments, the time interval between dosages is at least 6 weeks. In some embodiments, the time interval between dosages is at least 7 weeks. In some embodiments, the time interval between dosages is at least 8 weeks. Such dosages may be maintenance dosages.

In some embodiments a concentration of the compound 1, e.g. antisense oligomer such as miravirsen, in circulation in the subject, such as in the blood plasma, is maintained at a level of between 0.04 and 25 nM, such as between 0.8 and 20 nM.

In some embodiments, the dosage of the compound 1 administered at each dosing, such as unit dose, is within the range of 0.01 mg/kg-25 mg/kg. In some embodiments, the dosage, such as unit dose, of the compound administered at each dosing is within the range of 0.05 mg/kg-20 mg/kg. In some embodiments, the dosage (such as unit dose) of the compound administered at each dosing is within the range of 0.1 mg/kg-15 mg/kg. In some embodiments, the (such as unit dose) dosage of compound administered at each dosing is within the range of 1 mg/kg-15 mg/kg. In some embodiments, the dosage of the compound administered at each dosing is within the range of 1 mg/kg-10 mg/kg. In some embodiments, the dosage (such as unit dose) of the compound administered at each dosing is within the range of 0.01 mg/kg-25 mg/kg, such as about 0.01, 0.05, 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 16, 17, 18, 19, 20, 21, 22, 23, 24 or such as about 25 mg/kg, each of which are individual embodiments.

In some embodiments, compound 1, such as the oligomer, such as miravirsen, may be dosed at a range of 0.1 to 100 mg/kg, such as between 1 to 10 mg/kg or about 1 to about 12 mgs/kg. The dosing interval may, for example be between once a day and once every 2 months, such as between once a week, or once every two weeks and once a month or once every two months.

In some embodiments, the compositions of compound 1 (such as unit dose) are made for parenteral administration methods, such as in non limiting example, intra venous, sub cutaneous, intra peritoneal, intra cerebro vascular, intra nasal. In some embodiments, the administration is oral.

Suitably, such compositions comprise a pharmaceutically acceptable diluent, carrier, salt or adjuvant. PCT/DK2006/000512 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in PCT/DK2006/000512—which are also hereby incorporated by reference. In some embodiments, compound 1 is administered in water or saline water. In some embodiments, compound 1 is administered via a parenteral route of administration, such as intravenous or sub-cutaneous. In some embodiments, the administration route is via oral administration (see WO2011/048125, hereby incorporated by reference).

Compound 1 as used in the invention may be, in some embodiments, in a unit formulation (i.e. unit dose) such as in a pharmaceutically acceptable carrier or diluent in an amount sufficient to deliver to a patient a therapeutically effective amount without causing serious side effects in the treated patient.

The dosage of the pharmaceutical composition is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual oligonucleotides. Generally it can be estimated based on EC₅₀s found to be effective in in vitro and in vivo animal models. In general, dosage is from 0.01 μg to 1 g per kg of body weight, and may be given once or more daily, weekly, or monthly. The repetition rates for dosing can be estimated based on measured residence times and concentrations of the drug in bodily fluids or tissues.

### Reduced Standard of Care

The combination treatment according to the present invention may, in some embodiments, allow for a reduced treatment of interferon and ribavirin, or, in some embodiments, a treatment which is free from interferon and/or ribaviron treatment.

In some embodiments the period of interferon and/or ribavirin treatment is reduced to less than 48 weeks, such as less than 36 weeks, such as less than 24 weeks, or less than 12 weeks.

In some embodiments the reduced treatment of interferon and/or ribavirin may be in the form of a lower unit or daily dose.

### Examples of Compound 3 and Compound 4 Dosing in Standard of Care (Prior Art):

| Hepatitis C | Peg- | | | |
|---|---|---|---|---|
| Virus (HCV) | Interferon | | | Example |
| Genotype | Dose* | Ribavirin Dose | | Duration |
| Genotypes | 180 meg | <75 kg = | 1000 mg | 48 weeks |
| 1, 4 | | ≧75 kg = | 1200 mg | 48 weeks |

| | | | |
|---|---|---|---|
| Genotypes | 180 meg | 800 mg | 24 weeks |
| 2, 3 | | | |

In some embodiment the dosage of compound 4 (for example PEGASYS™) when used in the combination treatment according to the invention, or as part of the pre-treatment and/or post combination treatment period is less that 180 meg, such as less than 150 meg, such as less than 120 meg, such as less than 100 meg.

In some embodiment the dosage of compound 3 (for example COPEGUS™) when used in the combination treatment according to the invention, or as part of the pre-treatment and/or post combination treatment period is less than 800 mg, such as less than 700 mg, such as less than 600 mg, such as less than 500 mg, or less than 16 mg/kg, such as less than 14 mg/kg, such as less than 13 mg/kg, such as less than 12 mg/kg, such as less than 10 mg/kg, such as less than 8 mg/kg.

### Terms

The term “oligomer” in the context of the present invention, refers to a molecule formed by covalent linkage of two or more nucleotides (i.e. an oligonucleotide). Herein, a single nucleotide (unit) may also be referred to as a monomer or unit. In some embodiments, the terms “nucleoside”, “nucleotide”, “unit” and “monomer” are used interchangeably. It will be recognised that when referring to a sequence of nucleotides or monomers, what is referred to is the sequence of bases, such as A, T, G, C or U.

The oligomer typically consists or comprises of a contiguous nucleotide sequence of from 7-25 units.

In various embodiments, the compound of the invention does not comprise RNA (units). It is preferred that the compound according to the invention is a linear molecule or is synthesised as a linear molecule. The oligomer is a single stranded molecule, and preferably does not comprise short regions of, for example, at least 3, 4 or 5 contiguous nucleotides, which are complementary to equivalent regions within the same oligomer (i.e. duplexes)—in this regards, the oligomer is not (essentially) double stranded. In some embodiments, the oligomer is essentially not double stranded, such as is not a siRNA. In various embodiments, the oligomer of the invention may consist entirely of the contiguous nucleotide region. Thus, the oligomer is not substantially self-complementary.

The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

The terms “reverse complement”, “reverse complementary” and “reverse complementarity” as used herein are interchangeable with the terms “complement”, “complementary” and “complementarity”.

### Nucleosides and Nucleoside Analogues

In some embodiments, the terms “nucleoside analogue” and “nucleotide analogue” are used interchangeably.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogues” herein. Herein, a single nucleotide (unit) may also be referred to as a monomer or nucleic acid unit.

In field of biochemistry, the term “nucleoside” is commonly used to refer to a glycoside comprising a sugar moiety and a base moiety, and may therefore be used when referring to the nucleotide units, which are covalently linked by the internucleotide linkages between the nucleotides of the oligomer. In the field of biotechnology, the term “nucleotide” is often used to refer to a nucleic acid monomer or unit, and as such in the context of an oligonucleotide may refer to the base—such as the “nucleotide sequence”, typically refer to the nucleobase sequence (i.e. the presence of the sugar backbone and internucleoside linkages are implicit). Likewise, particularly in the case of oligonucleotides where one or more of the internucleoside linkage groups are modified, the term “nucleotide” may refer to a “nucleoside” for example the term “nucleotide” may be used, even when specifying the presence or nature of the linkages between the nucleosides.

As one of ordinary skill in the art would recognise, the 5′ terminal nucleotide of an oligonucleotide does not comprise a 5′ internucleotide linkage group, although may or may not comprise a 5′ terminal group.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1: The oligomer may thus comprise or consist of a simple sequence of natural occurring nucleotides—such as 2′-deoxynucleotides (referred to here generally as “DNA”), but also possibly ribonucleotides (referred to here generally as “RNA”), or a combination of such naturally occurring nucleotides and one or more non-naturally occurring nucleotides, i.e. nucleotide analogues. Such nucleotide analogues may suitably enhance the affinity of the oligomer for the target sequence.

Examples of suitable nucleotide analogues are provided by WO2007/031091 or are referenced therein.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

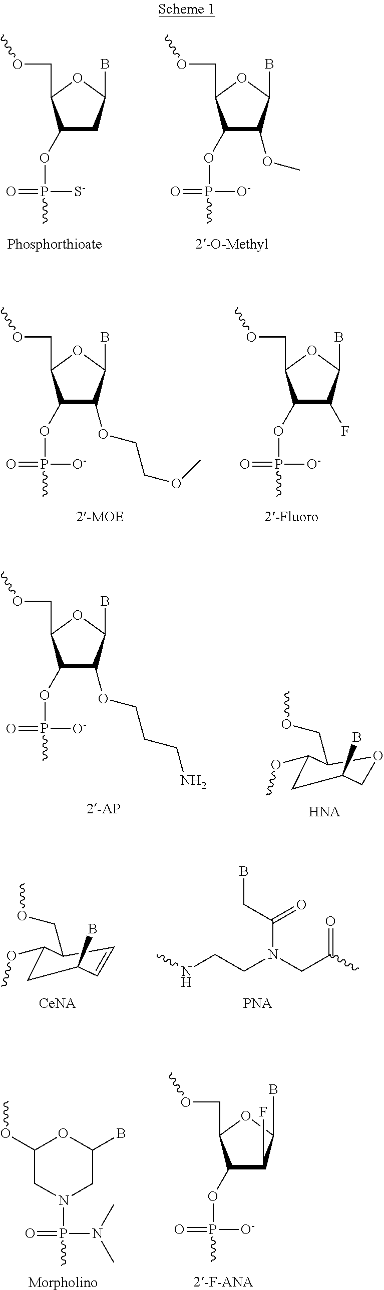

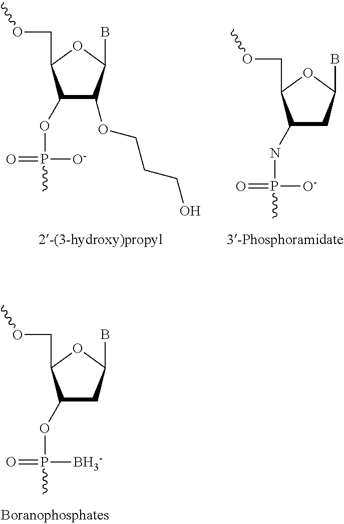

In some embodiments, the oligomer comprises at least 1 nucleoside analogue. In some embodiments the oligomer comprises at least 2 nucleotide analogues. In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

It will be recognised that when referring to a preferred nucleotide sequence motif or nucleotide sequence, which consists of only nucleotides, the oligomers of the invention which are defined by that sequence may comprise a corresponding nucleotide analogue in place of one or more of the nucleotides present in said sequence, such as LNA units or other nucleotide analogues, which raise the duplex stability/Tₘ of the oligomer/target duplex (i.e. affinity enhancing nucleotide analogues).

In some embodiments, any mismatches between the nucleotide sequence of the oligomer and the target sequence are preferably found in regions outside the affinity enhancing nucleotide analogues, such as region B as referred to herein, and/or region D as referred to herein, and/or at the site of non modified such as DNA nucleotides in the oligonucleotide, and/or in regions which are 5′ or 3′ to the contiguous nucleotide sequence.

Examples of such modification of the nucleotide include modifying the sugar moiety to provide a 2′-substituent group or to produce a bridged (locked nucleic acid) structure which enhances binding affinity and may also provide increased nuclease resistance.

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). Most preferred is beta-D-oxy-LNA.

In some embodiments the nucleotide analogues present within the oligomer of the invention (such as in regions A and C mentioned herein) are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid—Christensen, 2002. Nucl. Acids. Res. 2002 30: 4918-4925, hereby incorporated by reference) units and 2′MOE units. In some embodiments there is only one of the above types of nucleotide analogues present in the oligomer of the invention, or contiguous nucleotide sequence thereof.

In some embodiments the nucleotide analogues are 2′-O-methoxyethyl-RNA (2′MOE), 2′-fluoro-DNA monomers or LNA nucleotide analogues, and as such the oligonucleotide of the invention may comprise nucleotide analogues which are independently selected from these three types of analogue, or may comprise only one type of analogue selected from the three types. In some embodiments at least one of said nucleotide analogues is 2′-MOE-RNA, such as 2, 3, 4, 5, 6, 7, 8, 9 or 10 2′-MOE-RNA nucleotide units. In some embodiments at least one of said nucleotide analogues is 2′-fluoro DNA, such as 2, 3, 4, 5, 6, 7, 8, 9 or 10 2′-fluoro-DNA nucleotide units.

In some embodiments, the oligomer according to the invention comprises at least one Locked Nucleic Acid (LNA) unit, such as 1, 2, 3, 4, 5, 6, 7, or 8 LNA units, such as from 3-7 or 4 to 8 LNA units, or 3, 4, 5, 6 or 7 LNA units. In some embodiments, all the nucleotide analogues are LNA. In some embodiments, the oligomer may comprise both beta-D-oxy-LNA, and one or more of the following LNA units: thio-LNA, amino-LNA, oxy-LNA, and/or ENA in either the beta-D or alpha-L configurations or combinations thereof. In some embodiments all LNA cytosine units are 5′methyl-Cytosine. In some embodiments of the invention, the oligomer may comprise both LNA and DNA units. Preferably the combined total of LNA and DNA units is 10-25, such as 10-24, such as 10-20, such as 10-18, such as 12-16. In some embodiments of the invention, the nucleotide sequence of the oligomer, such as the contiguous nucleotide sequence consists of at least one LNA and the remaining nucleotide units are DNA units. In some embodiments the oligomer comprises only LNA nucleotide analogues and naturally occurring nucleotides (such as RNA or DNA, such as DNA nucleotides), optionally with modified internucleotide linkages such as phosphorothioate.

The term “nucleobase” refers to the base moiety of a nucleotide and covers both naturally occuring a well as non-naturally occurring variants. Thus, “nucleobase” covers not only the known purine and pyrimidine heterocycles but also heterocyclic analogues and tautomeres thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In some embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

### LNA

The term “LNA” refers to a bicyclic nucleoside analogue, known as “Locked Nucleic Acid”. It may refer to an LNA monomer, or, when used in the context of an “LNA oligonucleotide”, LNA refers to an oligonucleotide containing one or more such bicyclic nucleotide analogues. LNA nucleotides are characterised by the presence of a linker group (such as a bridge) between C2′ and C4′ of the ribose sugar ring—for example as shown as the biradical R^{4*}—R^{2*} as described below.

The LNA used in the oligonucleotide compounds of the invention preferably has the structure of the general formula I

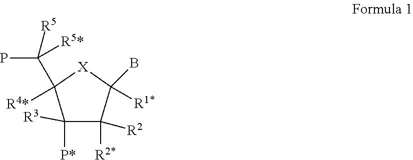

wherein for all chiral centers, asymmetric groups may be found in either R or S orientation;

wherein X is selected from —O—, —S—, —N(R^{N*})—, —C(R⁶R^{6*})—, such as, in some embodiments —O—;

B is selected from hydrogen, optionally substituted C₁₋₄-alkoxy, optionally substituted C₁₋₄-alkyl, optionally substituted C₁₋₄-acyloxy, nucleobases including naturally occurring and nucleobase analogues, DNA intercalators, photochemically active groups, thermochemically active groups, chelating groups, reporter groups, and ligands; preferably, B is a nucleobase or nucleobase analogue;

P designates an internucleotide linkage to an adjacent monomer, or a 5′-terminal group, such internucleotide linkage or 5′-terminal group optionally including the substituent R⁵ or equally applicable the substituent R^{5*};

P* designates an internucleotide linkage to an adjacent monomer, or a 3′-terminal group;

R^{4*} and R^{2*} together designate a bivalent linker group consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z, wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, optionally substituted C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), wherein for all chiral centers, asymmetric groups may be found in either R or S orientation, and;

each of the substituents R^{1*}, R², R³, R⁵, R^{5*}, R⁶ and R^{6*}, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene; wherein R^{N} is selected from hydrogen and C₁₋₄-alkyl, and where two adjacent (non-geminal) substituents may designate an additional bond resulting in a double bond; and R^{N*}, when present and not involved in a biradical, is selected from hydrogen and C₁₋₄-alkyl; and basic salts and acid addition salts thereof. For all chiral centers, asymmetric groups may be found in either R or S orientation.

In some embodiments, R^{4*} and R^{2*} together designate a biradical consisting of a groups selected from the group consisting of C(R^{a}R^{b})—C(R^{a}R^{b})—, C(R^{a}R^{b})—O—, C(R^{a}R^{b})—NR^{a}—, C(R^{a}R^{b})—S—, and C(R^{a}R^{b})—C(R^{a}R^{b})—O—, wherein each R^{a} and R^{b} may optionally be independently selected. In some embodiments, R^{a} and R^{b} may be, optionally independently selected from the group consisting of hydrogen and _{C1-6}alkyl, such as methyl, such as hydrogen.

In some embodiments, R^{4*} and R^{2*} together designate the biradical —O—CH(CH₂OCH₃)— (2′O-methoxyethyl bicyclic nucleic acid—Seth at al., 2010, J. Org. Chem)—in either the R- or S-configuration.

In some embodiments, R^{4*} and R^{2*} together designate the biradical —O—CH(CH₂CH₃)— (2′O-ethyl bicyclic nucleic acid—Seth at al., 2010, J. Org. Chem). —in either the R- or S-configuration.

In some embodiments, R^{4*} and R^{2*} together designate the biradical —O—CH(CH₃)—. —in either the R- or S-configuration. In some embodiments, R^{4*} and R^{2*} together designate the biradical —O—CH₂—O—CH₂——(Seth at al., 2010, J. Org. Chem).

In some embodiments, R^{4*} and R^{2*} together designate the biradical —O—NR—CH₃——(Seth at al., 2010, J. Org. Chem).

In some embodiments, the LNA units have a structure selected from the following group:

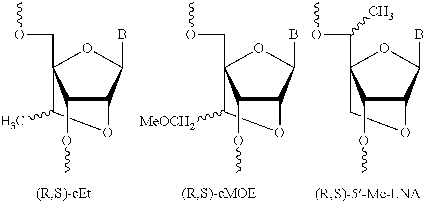

In some embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆alkynyl or substituted C₂₋₆alkynyl, C₁₋₆alkoxyl, substituted C₁₋₆alkoxyl, acyl, substituted acyl, C₁₋₆aminoalkyl or substituted C₁₋₆ aminoalkyl. For all chiral centers, asymmetric groups may be found in either R or S orientation.

In some embodiments, R^{1*}, R², R³, R⁵, R^{5*} are hydrogen.

In some embodiments, R^{1*}, R², R³ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆alkynyl or substituted C₂₋₆alkynyl, alkoxyl, substituted C₁₋₆alkoxyl, acyl, substituted acyl, C₁₋₆aminoalkyl or substituted C₁₋₆ aminoalkyl. For all chiral centers, asymmetric groups may be found in either R or S orientation.

In some embodiments, R^{1*}, R², R³ are hydrogen.

In some embodiments, R⁵ and R^{5*} are each independently selected from the group consisting of H, —CH₃, —CH₂—CH₃, —CH₂—O—CH₃, and —CH═CH₂. Suitably in some embodiments, either R⁵ or R^{5*} are hydrogen, where as the other group (R⁵ or R^{5*} respectively) is selected from the group consisting of C₁₋₅ alkyl, C₂₋₆ alkenyl, C₂₋₆alkynyl, substituted C₁₋₆ alkyl, substituted C₂₋₆ alkenyl, substituted C₂₋₆alkynyl or substituted acyl (—C(═O)—); wherein each substituted group is mono or poly substituted with substituent groups independently selected from halogen, C₁₋₆ alkyl, substituted C₁₋₆ alkyl, C₂₋₆ alkenyl, substituted C₂₋₆ alkenyl, C₂₋₆alkynyl, substituted C₂₋₆alkynyl, OJ₁, SJ₁, NJ₁J₂, N₃, COOJ₁, CN, O—C(C═O)NJ₁J₂, N(H)C(═NH)NJ, J₂ or N(H)C(═X)N(H)J₂ wherein X is O or S; and each J₁ and J₂ is, independently, H, C₁₋₆ alkyl, substituted C₁₋₆ alkyl, C₂₋₆ alkenyl, substituted C₂₋₆ alkenyl, C₂₋₆alkynyl, substituted C₂₋₆alkynyl, C₁₋₆aminoalkyl, substituted C₁₋₆aminoalkyl or a protecting group. In some embodiments either R⁵ or R^{5*} is substituted C₁₋₆alkyl. In some embodiments either R⁵ or R^{5*} is substituted methylene wherein preferred substituent groups include one or more groups independently selected from F, NJ₁J₂, N₃, CN, OJ₁, SJ₁, O—C(═O)NJ₁J₂, N(H)C(═NH)NJ, J₂ or N(H)C(O)N(H)J₂. In some embodiments each J₁ and J₂ is, independently H or C₁₋₆ alkyl. In some embodiments either R⁵ or R^{5*} is methyl, ethyl or methoxymethyl. In some embodiments either R⁵ or R^{5*} is methyl. In a further embodiment either R⁵ or R^{5*} is ethylenyl. In some embodiments either R⁵ or R^{5*} is substituted acyl. In some embodiments either R⁵ or R^{5*} is C(═O)NJ₁J₂. For all chiral centers, asymmetric groups may be found in either R or S orientation. Such 5′ modified bicyclic nucleotides are disclosed in WO 2007/134181, which is hereby incorporated by reference in its entirety.

In some embodiments B is a nucleobase, including nucleobase analogues and naturally occurring nucleobases, such as a purine or pyrimidine, or a substituted purine or substituted pyrimidine, such as a nucleobase referred to herein, such as a nucleobase selected from the group consisting of adenine, cytosine, thymine, adenine, uracil, and/or a modified or substituted nucleobase, such as 5-thiazolo-uracil, 2-thio-uracil, 5-propynyl-uracil, 2′thio-thymine, 5-methyl cytosine, 5-thiozolo-cytosine, 5-propynyl-cytosine, and 2,6-diaminopurine.

In some embodiments, R^{4*} and R^{2*} together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}R^{b})—C(R^{c}R^{d})—S—, wherein R^{a}, R^{b}, R^{c}, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂). For all chiral centers, asymmetric groups may be found in either R or S orientation.

In a further embodiment R^{4*} and R^{2*} together designate a biradical (bivalent group) selected from —CH₂—O—, —CH₂—S—, —CH₂—NH—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, and —CH(CH₂—O—CH₃)—O—, and/or, —CH₂—CH₂—, and —CH═CH— For all chiral centers, asymmetric groups may be found in either R or S orientation.

In some embodiments, R^{4*} and R^{2*} together designate the biradical C(R^{a}R^{b})—N(R^{c})—O—, wherein R^{a} and R^{b} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen, and; wherein R^{c} is selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R^{4*} and R^{2*} together designate the biradical C(R^{a}R^{b})—O—C(R^{c}R^{d})—O—, wherein R^{a}, R^{b}, R^{c}, and R^{d} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R^{4*} and R^{2*} form the biradical —CH(Z)—O—, wherein Z is selected from the group consisting of C₁₋₆ alkyl, C₂₋₆ alkenyl, C₂₋₆ alkynyl, substituted C₁₋₆ alkyl, substituted C₂₋₆ alkenyl, substituted C₂₋₆ alkynyl, acyl, substituted acyl, substituted amide, thiol or substituted thio; and wherein each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ³C(═X)NJ₁J₂ and CN, wherein each J₁, J₂ and J₃ is, independently, H or C₁₋₆ alkyl, and X is O, S or NJ₁. In some embodiments Z is C₁₋₆ alkyl or substituted C₁₋₆ alkyl. In some embodiments Z is methyl. In some embodiments Z is substituted C₁₋₆ alkyl. In some embodiments said substituent group is C₁₋₆alkoxy. In some embodiments Z is CH₃OCH₂—. For all chiral centers, asymmetric groups may be found in either R or S orientation. Such bicyclic nucleotides are disclosed in U.S. Pat. No. 7,399,845 which is hereby incorporated by reference in its entirety. In some embodiments, R^{1*}, R², R³, R⁵, R^{5*} are hydrogen. In some embodiments, R^{1*}, R², R^{3*} are hydrogen, and one or both of R⁵, R^{5*} may be other than hydrogen as referred to above and in WO 2007/134181.

In some embodiments, R^{4*} and R^{2*} together designate a biradical which comprise a substituted amino group in the bridge such as consist or comprise of the biradical —CH₂—N(R^{c})—, wherein R^{c} is C₁₋₁₂ alkyloxy. In some embodiments R^{4*} and R^{2*} together designate a biradical —Cq₃q₄-NOR—, wherein q₃ and q₄ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl; wherein each substituted group is, independently, mono or poly substituted with substituent groups independently selected from halogen, OJ₁, SJ₁, NJ₁J₂, COOJ₁, CN, O—C(═O)NJ₁J₂, N(H)C(═NH)NJ₁J₂ or N(H)C(═X═N(H)J₂ wherein X is O or S; and each of J₁ and J₂ is, independently, H, C₁₋₆ alkyl, C₂₋₆ alkenyl, C₂₋₆ alkynyl, C₁₋₆ aminoalkyl or a protecting group. For all chiral centers, asymmetric groups may be found in either R or S orientation. Such bicyclic nucleotides are disclosed in WO2008/150729 which is hereby incorporated by reference in its entirety. In some embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆alkoxyl, substituted C₁₋₆alkoxyl, acyl, substituted acyl, C₁₋₆ aminoalkyl or substituted C₁₋₆ aminoalkyl. In some embodiments, R^{1*}, R², R³, R⁵, R^{5*} are hydrogen. In some embodiments, R^{1*}, R², R³ are hydrogen and one or both of R⁵, R^{5*} may be other than hydrogen as referred to above and in WO 2007/134181. In some embodiments R^{4*} and R^{2*} together designate a biradical (bivalent group) C(R^{a}R^{b})—O—, wherein R^{a} and R^{b} are each independently halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ₁SJ₁, SOJ₁, SO₂J₁, NJ₁J₂, N₃, CN, C(═O)OJ₁, C(═O)NJ₁J₂, C(═O)J₁, O—C(═O)NJ₁J₂, N(H)C(═NH)NJ₁J₂, N(H)C(═O)NJ₁J₂ or N(H)C(═S)NJ₁J₂; or R^{a} and R^{b} together are ═C(q3)(q4); q₃ and q₄ are each, independently, H, halogen, C₁-C₁₂alkyl or substituted C₁-C₁₂ alkyl; each substituted group is, independently, mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ₁J₂, N₃, CN, C(═O)OJ₁, C(═O)NJ₁J₂, C(═O)J₁, O—C(═O)NJ₁J₂, N(H)C(═O)NJ₁J₂ or N(H)C(═S)NJ₁J₂ and; each J₁ and J₂ is, independently, H, C1-C₆ alkyl, substituted C1-C₆ alkyl, C₂-C₆ alkenyl, substituted C₂-C₆ alkenyl, C₂-C₆ alkynyl, substituted C₂-C₆ alkynyl, C1-C₆ aminoalkyl, substituted C1-C₆ aminoalkyl or a protecting group. Such compounds are disclosed in WO2009006478A, hereby incorporated in its entirety by reference.

In some embodiments, R^{4*} and R^{2*} form the biradical -Q-, wherein Q is C(q₁)(q₂)C(q₃)(q₄), C(q₁)═C(q₃), C[═C(q₁)(q₂)]—C(q₃)(q₄) or C(q₁)(q₂)—C[=(q₃)(q₄)]; q₁, q₂, q₃, q₄ are each independently. H, halogen, C₁₋₁₂ alkyl, substituted C₁₋₁₂ alkyl, C₂₋₁₂ alkenyl, substituted C₁₋₁₂ alkoxy, OJ₁, SJ₁, SOJ₁, SO₂J₁, NJ₁J₂, N₃, CN, C(═O)OJ₁, C(═O)—NJ₁J₂, C(═O) J₁, —C(C═O)NJ₁J₂, N(H)C(═NH)NJ₁J₂, N(H)C(═O)NJ₁J₂ or N(H)C(═S)NJ₁J₂; each J₁ and J₂ is, independently, H, C₁₋₆ alkyl, C₂₋₆ alkenyl, C₂₋₆ alkynyl, C₁₋₆ aminoalkyl or a protecting group; and, optionally wherein when Q is C(q₁)(q₂)(q₃)(q₄) and one of q₃ or q₄ is CH₃ then at least one of the other of q₃ or q₄ or one of q₁ and q₂ is other than H. In some embodiments, R^{1*}, R², R³, R⁵, R^{5*} are hydrogen. For all chiral centers, asymmetric groups may be found in either R or S orientation. Such bicyclic nucleotides are disclosed in WO2008/154401 which is hereby incorporated by reference in its entirity. In some embodiments, R^{1*}, R², R³, R⁵, R^{5*} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆alkoxyl, acyl, substituted acyl, C₁₋₆aminoalkyl or substituted C₁₋₆ aminoalkyl. In some embodiments, R^{1*}, R², R³, R⁵, R^{5*} are hydrogen. In some embodiments, R^{1*}, R², R³ are hydrogen and one or both of R⁵, R^{5*} may be other than hydrogen as referred to above and in WO 2007/134181 or WO2009/067647 (alpha-L-bicyclic nucleic acids analogs).

In some embodiments the LNA used in the oligonucleotide compounds of the invention preferably has the structure of the general formula II:

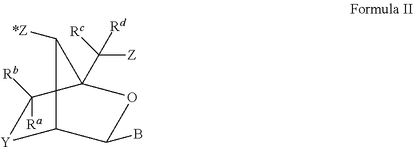

wherein Y is selected from the group consisting of —O—, —CH₂O—, —S—, —NH—, N(R^{e}) and/or —CH₂—; Z and Z* are independently selected among an internucleotide linkage, R^{H}, a terminal group or a protecting group; B constitutes a natural or non-natural nucleotide base moiety (nucleobase), and R^{H} is selected from hydrogen and C₁₋₄-alkyl; R^{a}, R^{b} R^{c}, R^{d} and R^{e} are, optionally independently, selected from the group consisting of hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂); and R^{H} is selected from hydrogen and C₁₋₄-alkyl. In some embodiments R^{a}, R^{b} R^{c}, R^{d} and R^{e} are, optionally independently, selected from the group consisting of hydrogen and C₁₋₆ alkyl, such as methyl. For all chiral centers, asymmetric groups may be found in either R or S orientation, for example, two exemplary stereochemical isomers include the beta-D and alpha-L isoforms, which may be illustrated as follows:

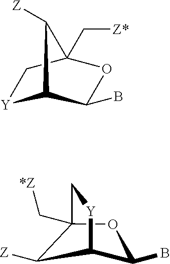

Specific exemplary LNA units are shown below:

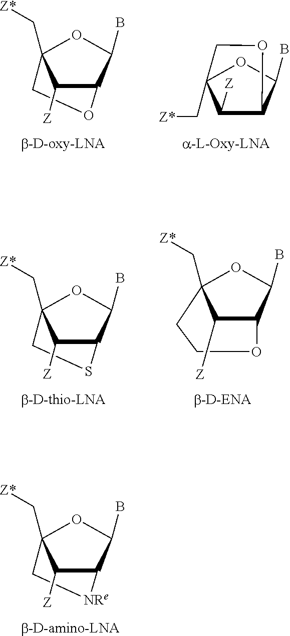

The term “thio-LNA” comprises a locked nucleotide in which Y in the general formula above is selected from S or —CH₂—S—. Thio-LNA can be in both beta-D and alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in the general formula above represents —O—. Oxy-LNA can be in both beta-D and alpha-L-configuration.

The term “ENA” comprises a locked nucleotide in which Y in the general formula above is —CH₂—O— (where the oxygen atom of —CH₂—O— is attached to the 2′-position relative to the base B). R^{e} is hydrogen or methyl.

In some exemplary embodiments LNA is selected from beta-D-oxy-LNA, alpha-L-oxy-LNA, beta-D-amino-LNA and beta-D-thio-LNA, in particular beta-D-oxy-LNA.

### Internucleotide Linkages

The monomers of the oligomers described herein are coupled together via linkage groups. Suitably, each monomer is linked to the 3′ adjacent monomer via a linkage group.

The person having ordinary skill in the art would understand that, in the context of the present invention, the 5′ monomer at the end of an oligomer does not comprise a 5′ linkage group, although it may or may not comprise a 5′ terminal group.

The terms “linkage group” or “internucleotide linkage” are intended to mean a group capable of covalently coupling together two nucleotides. Specific and preferred examples include phosphate groups and phosphorothioate groups.

The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within WO2007/031091, for example the internucleotide linkages listed on the first paragraph of page 34 of WO2007/031091 (hereby incorporated by reference).

It is, in some embodiments, preferred to modify the internucleotide linkage from its normal phosphodiester to one that is more resistant to nuclease attack, such as phosphorothioate or boranophosphate.

Suitable sulphur (S) containing internucleotide linkages as provided herein may be preferred. Phosphorothioate internucleotide linkages are also preferred.

In some embodiments, such as the embodiments referred to above, where suitable and not specifically indicated, all remaining linkage groups are either phosphodiester or phosphorothioate, or a mixture thereof.

In some embodiments all the internucleotide linkage groups are phosphorothioate.

### Conjugates

In the context the term “conjugate” is intended to indicate a heterogenous molecule formed by the covalent attachment (“conjugation”) of the oligomer as described herein to one or more non-nucleotide, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol.

Therefore, in various embodiments, the oligomer of the invention may comprise both a polynucleotide region which typically consists of a contiguous sequence of nucleotides, and a further non-nucleotide region. When referring to the oligomer of the invention consisting of a contiguous nucleotide sequence, the compound may comprise non-nucleotide components, such as a conjugate component.

In various embodiments of the invention the oligomeric compound is linked to ligands/conjugates, which may be used, e.g. to increase the cellular uptake of oligomeric compounds. WO2007/031091 provides suitable ligands and conjugates, which are hereby incorporated by reference.

The invention also provides for a conjugate comprising the compound according to the invention as herein described, and at least one non-nucleotide or non-polynucleotide moiety covalently attached to said compound. Therefore, in various embodiments where the compound of the invention consists of a specified nucleic acid or nucleotide sequence, as herein disclosed, the compound may also comprise at least one non-nucleotide or non-polynucleotide moiety (e.g. not comprising one or more nucleotides or nucleotide analogues) covalently attached to said compound.

Conjugation (to a conjugate moiety) may enhance the activity, cellular distribution or cellular uptake of the oligomer of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

The oligomers of the invention may also be conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of, for example from 1-50, such as 2-20 such as 3-10 amino acid residues in length, and/or polyalkylene oxide such as polyethylglycol(PEG) or polypropylene glycol—see WO 2008/034123, hereby incorporated by reference. Suitably the positively charged polymer, such as a polyalkylene oxide may be attached to the oligomer of the invention via a linker such as the releasable inker described in WO 2008/034123.

By way of example, the following conjugate moieties may be used in the conjugates of the invention:

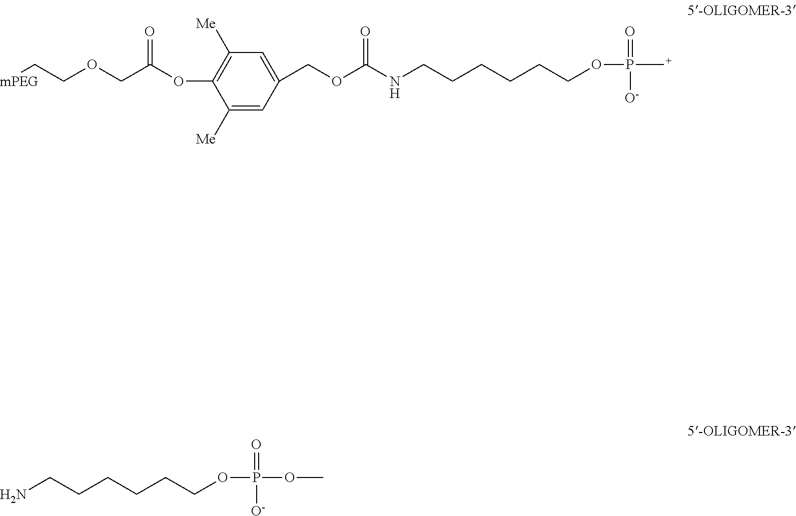

### Activated Oligomers

The term “activated oligomer,” as used herein, refers to an oligomer of the invention that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that is preferably hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl. In some embodiments, the functional moiety is self-cleaving. In other embodiments, the functional moiety is biodegradable. See e.g., U.S. Pat. No. 7,087,229, which is incorporated by reference herein in its entirety.

In some embodiments, oligomers of the invention are functionalized at the 5′ end in order to allow covalent attachment of the conjugated moiety to the 5′ end of the oligomer. In other embodiments, oligomers of the invention can be functionalized at the 3′ end. In still other embodiments, oligomers of the invention can be functionalized along the backbone or on the heterocyclic base moiety. In yet other embodiments, oligomers of the invention can be functionalized at more than one position independently selected from the 5′ end, the 3′ end, the backbone and the base.

In some embodiments, activated oligomers of the invention are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis. In some embodiments, the oligomers are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, preferably about 6, wherein the alkyl portion of the alkylamino group can be straight chain or branched chain, and wherein the functional group attached to the oligomer via an ester group (—O—C(O)—(CH₂)_{w}SH)

In some embodiments, sulfhydryl-activated oligonucleotides are conjugated with polymer moieties such as polyethylene glycol or peptides (via formation of a disulfide bond).

Activated oligomers containing hindered esters as described above can be synthesized by any method known in the art, and in particular by methods disclosed in PCT Publication No. WO 2008/034122 and the examples therein, which is incorporated herein by reference in its entirety.

In still other embodiments, the oligomers of the invention are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers of the invention have a functionalizing reagent coupled to a hydroxyl group on the backbone of the oligomer. In yet further embodiments, the oligomer of the invention is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(0-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 1991, 34, 7171.

In still further embodiments, the oligomers of the invention may have amine-containing functional moieties on the nucleobase, including on the N6 purine amino groups, on the exocyclic N2 of guanine, or on the N4 or 5 positions of cytosine. In various embodiments, such functionalization may be achieved by using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.). In some embodiments in some embodiments

### Compositions

The oligomer of the invention may be used in pharmaceutical formulations and compositions. Suitably, such compositions comprise a pharmaceutically acceptable diluent, carrier, salt or adjuvant. PCT/DK2006/000512 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in PCT/DK2006/000512—which are also hereby incorporated by reference.

### FURTHER EMBODIMENTS

1. A method for the treatment of hepatitis C (HCV) infection in a subject infected with HCV, said method comprising the steps of administering an effective amount of a miR-122 inhibitor and an effective amount of a HCV NS3/4A protease inhibitor to the subject infected with HCV.
2. The method according to embodiment 1, wherein said miR-122 inhibitor is selected from the group consisting of an antisense oligomer and a small molecule inhibitor of miR-122.
3. The method according to embodiment 1 or 2, wherein the miR-122 inhibitor is an antisense oligomer which is fully complementary to the mature hsa-miR-122 sequence (SEQ ID NO 1) across the entire length of the oligonucleotide.
4. The method according to embodiment 3 wherein the antisense oligomer is a mixmer or a totalmer.
5. The method according to any one of embodiments 2-4, wherein the oligomer is 7-18 contiguous nucleotides in length.
6. The method according to any one of embodiments 2-5, wherein the oligomer comprises non-naturally occurring nucleotides or nucleotide other than DNA or RNA.
7. The method according to any one of embodiments 2-6, wherein the oligomer comprises nucleotide analogues.
8. The method according to embodiment 7, wherein the nucleotide analogues are sugar modified nucleotides, such as sugar modified nucleotides, optionally independently, selected from the group consisting of: Locked Nucleic Acid (LNA) units; 2′-O-alkyl-RNA units, 2′-OMe-RNA units, 2′-amino-DNA units, optionally substituted hexitol nucleic acid unit, and 2′-fluoro-DNA units.
9. The method according to embodiment 8, wherein the nucleotide analogues are LNA.
10. The method according to one of embodiments 1-9 which is essentially incapable of recruiting RNaseH.
11. The method according to any one of embodiments 1-0.9 which is a mixmer or a totalmer.
12. The method according to any one of embodiments 1-11 which has a length of 7, 8, 9 or 10 nucleobases.
13. The method according to any one of embodiments 1-11, which has a length of 10, 11, 12, 13, 14, 15 or 16 nucleobases.
14. The method according to any one of embodiments 1-13, which comprises of DNA and nucleotide analogue nucleobases, or only nucleotide analogue nucleobases.
15. The method according to any one of embodiments 1-14, which does not comprise a region of more than 4, or more than 3, or more than 2 consecutive DNA nucleotides.
16. The method according to any one of embodiments 1-15 in which all the nucleotides of the oligomer are nucleotide analogues.
17. The method according to any one of embodiments 1-16, wherein all the nucleotides of the oligomer are LNA nucleotides.
18. The method according to any one of embodiments 1-17, wherein the internucleoside linkages are, optionally independently, selected from the group consisting of phosphorotyhioate and phosphodiester.
19. The method according to any one of embodiments 1-18, wherein the oligomer comprises at least on phosphorothiate linkage or all the internucleoside linkages are phosphorothioate linkages.
20. The method according to any one of embodiments 1-19, wherein the contiguous nucleotide sequence of the oligomer is selected from the sequences listed herein.
21. The method according to any one of embodiments 1-19, wherein the oligomer consists or comprises the formula:

5′-^{m}Cₛ^{o}cₛAₛ^{o}tₛtₛGₛ^{o}Tₛ^{o}cₛaₛ^{m}Cₛ^{o}aₛ^{m}Cₛ^{o}tₛ^{m}Cₛ^{om}C^{o}-3′
wherein; a lowercase letter identifies a DNA unit, and an upper case letter identifies a LNA unit, ^{m}C identifies a 5-methylcytosine LNA, subscript ₛ identifies a phosphorothioate internucleoside linkage, and wherein LNA units are beta-D-oxy, as identified by a ^{o} superscript after LNA residue.
22. The method according to any one of embodiments 1-21, wherein the HCV NS3/4A protease inhibitor is selected from the group consisting of: Tegobuvir, Narlaprevir, Vaniprevir, Danoprevir, Filibuvir, Telaprevir, Coceprevir and Boceprevir.
23. The method according any one of embodiments 1-21, wherein the HCV NS3/4A protease inhibitor is Telaprevir or Boceprevir.
24. The method according to any one of embodiments 1-23 wherein the treatment is interferon free.
25. The method according to any one of embodiments 1-23 wherein the treatment is in combination with interferon and optionally ribavirin treatment.
26. The method according to any one of embodiments 1-25, wherein multiple doses of the miR-122 inhibitor and multiple doses of the HCV NS3/4A protease inhibitor, and optionally ribavirin, are administered over a treatment period of less than 1 year.
27. The method according to embodiment 26 wherein the duration of the treatment period, is less than 48 weeks, such as less than 24 weeks, or less than 13 weeks.
28. The method according to any one of embodiments 1-27, wherein the subject is an interferon-non responder.
29. The method according to any one of embodiments 1-28, wherein the HCV is selected genotypes 1, 2, 3, 4, 5, and 6, such as genotype 1a and/or genotype 1 b.
30. The method according to any one of embodiments 1-29, wherein the subject has chronic hepatitis C (CHC).
31. The method according to any one of embodiments 1-30, wherein the subject co-infected with both HIV and HCV.
32. The method according to any one of embodiments 1-31, wherein the subject is an interferon non-responder, a partial responder, a relapse responder or a null responder.
33. The method according to any one of embodiments 1-32, wherein the miR-122 inhibitor is administered to the subject in a dose of between about 0.1 mg/kg and 10 mg/kg.
34. The method according to any one of embodiments 1-33, wherein the at least two separate doses of the miR-122 inhibitor are administered and the time interval between successive doses miR-122 inhibitor is daily, weekly or monthly, or a time interval between daily and monthly.
35. A method of reducing the level of HCV infection in a cell, said method comprising contacting a cell infected with a HCV with a miR-122 inhibitor and at least one HCV NS3/4A protease inhibitor.
36. The use of a miR-122 inhibitor for the preparation of a medicament for the treatment of Hepatitis C, wherein said medicament is for use in combination with an HCV NS3/4A protease inhibitor.
37. A miR-122 inhibitor for use in the treatment of Hepatitis C in combination with an HCV NS3/4A protease inhibitor.

### EXAMPLES

### Example 1

Miravirsen (SPC3649) was tested alone to determine the EC₅₀ (efficacy) and CC₅₀ (cellular toxicity) values. The EC₅₀ and CC₅₀ values for miravirsen are presented in the table below. In vitro EC₅₀ and CC5₀ determination for miravirsen (SPC3649)

| Compound | EC₅₀ | (+/− SD) | CC₅₀ | (+/−SD) |
|---|---|---|---|---|
| miravirsen | 0.67 μM | (+/−0.33) | >158 μM | (+/−na) |

The anti-viral efficacy and cellular toxicity of non-transfected antimiR oligonucleotide in combination with approved and experimental anti-HCV therapeutics (boceprevir or telepravir, was determined in the reporter cell line Huh-luc/neo-ET. This cell line harbors the persistently replicating I₃₈₉luc-ubi-neo/NS3-3′/ET replicon containing the firefly luciferase gene-ubiquitin-neomycin phosphotransferase fusion protein and EMCV IRES driven NS3-5B HCV coding sequences containing the ET tissue culture adaptive mutations (E1202G, T1208I, and K1846T). Eight dilutions of non-transfected antimiR oligonucleotide (bracketing the calculated EC₅₀) were evaluated in triplicate on duplicate sets of three plates with five dilutions of each of the control compounds (bracketing the calculated EC₅₀). One set of plates was used for the determination of cellular toxicity and the other set of plates is used for the determination of anti-viral efficacy. The efficacy and toxicity results was quantified with firefly luciferase activity and XTT dye reduction, respectively, and the results of the assay is imported into the Prichard and Shipman MacSynergy II software program. The MacSynery II analysis includes calculation of the dose response of the two (or more) compounds when used alone followed by calculation of the expected additive level of antiviral inhibition or toxicity when the compounds are used together based on the individual dose response curves. The expected level of activity at each of the concentration data points is compared to the experimentally determined antiviral activity in the assay. The expected activity was subtracted from the realized activity, yielding a negative, zero or positive value. These values are plotted as a three dimensional representation of the data and the result is reported as a two-factor value, expressing the combined average above the expected level of activity (synergy volume) and the combined average below the expected level of activity (antagonism volume)Combination analysis was performed two or more times for each pair of test compound and control substance.

### Example 2

### Anti-HCV Evaluations of Non-Transfected SPC3649 Anti-miR Oligonucleotide in Combination with Interferon-a2b, Ribavirin, 2′-Methylcytidine, VX-222, BMS-790052, and Telaprevir in HCV Genotype I b Replicon Cells

This example was based on an in vitro evaluation of SPC3649 (miravirsen) in combination with anti-HCV drugs and experimental compounds. SPC3649 was evaluated in combination with six drugs/compounds representing various classes of antiviral activities, including interferon, ribavirin, NS3/4A protease inhibitor telaprevir, nucleoside NS5B inhibitor 2′-methylcytidine, non-nucleoside NS5B inhibitor VX-222, and NS5A inhibitor BMS-790052. The combination antiviral assays were performed using the reporter cell line Huh-luc/neo-ET, which contains a bicistronic HCV genotype Ib replicon. Combination data were analyzed using MacSynergy II software at the 95%, confidence interval. The in vitro combination assays were designed to define the antiviral interaction of the two compounds and to determine if their interaction was synergistic or antagonistic.

Interferon-a2b (IFN-a2b) was purchased from R&D Systems (Minneapolis, Minn.). Ribavirin (RBV) was purchased from Sigma-Aldrich (St. Louis, Mo.). Telaprevir, VX-222, and BMS-790052 were purchased from Selleck Chemicals (Houston, Tex.). 2′Methyl-Cytidine (2-MeC) was purchased from Toronto Research Chemicals (North York, Ontario, Canada).

### Cell Preparation

The reporter cell line Huh-luc/neo-ET was obtained from Dr. Ralf Bartenschlager (Department of Molecular Virology, Hygiene Institute, University of Heidelberg, Germany) by ImQuest BioSciences. This cell line harbors the persistently replicating 1389luc-ubi-neo/NS3-3′IET replicon containing the firefly luciferase gene-ubiquitinneomycin phosphotransferase fusion protein and EMCV IRES driven NS3-5B HCV coding sequences containing the ET tissue culture adaptive mutations (E 1202G, T12081, and KI846T). A stock culture of the Huh-luc/neo-ET was expanded by culture in DMEM supplemented with 10% FBS, 2 mM glutamine, penicillin (100 IU/mL)/streptomycin (100 f.Ig/mL) and IX nonessential amino acids plus I mg/ml G418. Prior to plating, the cells were split 1:4 and cultured for two passages in the same medium plus 250 f.Ig/mL G418. The cells were treated with trypsin and enumerated by staining with trypan blue and seeded into 96-well tissue culture plates at a cell culture density 5.0×103 cells per well in a volume of 85 f.IL per well and incubated at 3ic in an environment of 5% CO2 for 24 hours. Six plates were established for the determination of combination efficacy (EC₅₀) and cytotoxicity (TC₅₀) for each of the compound combinations (three plates each for efficacy and toxicity). Following the 24 hour incubation, eight 2-fold serial dilutions of SPC3649 in cell culture medium without G418 were prepared (final in well high test concentration of 1.20 μM to 2.40 μM) and added to the cells and five 2-fold or 5-fold serial dilutions of control compounds in cell culture medium without G418 were prepared and added to the cells. The final in well high test concentrations, dilution schemes, and concentration range for each of the control compounds is displayed in Table A. Six wells in each plate also received medium alone as a no-treatment control. Separate duplicate plates were prepared in which IFN-a2b was added to triplicate wells at final concentrations of 10.0, 2.00, 0.400, 0.0800, 0.0160, and 0.0032 units per milliliter (U/ml) as a positive single compound control for antiviral efficacy. The cells were incubated for 48 hours at 37° C. in an environment of 5% CO₂. Following incubation, the plates were assessed for anti-HCV activity by measurement of luciferase reporter activity and cellular cytotoxicity by XTT staining.

Concentration of test compounds used for anti-HCV
combination analysis.

| Compound | | | | | |
|---|---|---|---|---|---|
| Tested in | | | | | |
| Combination | | | | | |
| with | High Test | | | | |
| SPC3649-09: | Concentration | | Dilution | Concentration Range | |
| Interferon-α2b | 10.0 | U/mL | 1:5 | 0.00320 to | 10.0 U/mL |
| VX 222 | 10.0 | nM | 1:5 | 16.0 pM to | 10.0 nM |
| 2-Me-C | 4.50 | nM | 1:5 | 7.20 pM to | 4.50 μM |
| Ribavirin | 148 | μM | 1:5 | 0.237 to | 148 μM |
| (1:5 dilution) | | | | | |
| Ribavirin | 148 | μM | 1:5 | 9.25 to | 148 μM |
| (1:2 dilution) | | | | | |
| BMS790052 | 32.0 | pM | 1:5 | 0.0510 to | 32.0 pM |
| (1:5 dilution) | | | | | |
| BMS90052 | 32.0 | pM | 1:5 | 2.00 to | 32.0 pM |
| (1:2 dilution) | | | | | |
| Telaprevir | 1.00 | μM | 1:5 | 1.60 nM to | 1.00 pM |
| (1:5 dilution) | | | | | |
| Telaprevir | 1.00 | μM | 1:5 | 62.5 nM to | 1.00 μM |
| (1:2 dilution) | | | | | |

### Measurement of Virus Replication:

HCV replication from the replicon assay system was measured by luciferase activity using the britelite plus luminescence reporter gene kit according to the manufacturer's instructions (Perkin Elmer, Shelton, Conn.). Briefly, one vial of britelite plus lyophilized substrate was solubilized in I0 mL of britelite reconstitution buffer and mixed gently by inversion. After a 5 minute incubation at room temperature, the britelite plus reagent was added to the 96 well plates at 100 μl per well. The well contents were transferred to a white 96-well plate and luminescence was measured within 15 minutes using the Wallac 1450 Microbeta Trilux liquid scintillation counter. The data for combination analysis were imported into a Prichard and Shipman (Antiviral Research 14:181-206 [1990]) MacSynergy II software template for analysis as described below. The data for the single compound IFN-a2b antiviral control were imported into a customized Microsoft Excel workbook for determination of the 50% virus inhibition concentration (EC₅₀).

### Cytotoxicity:

The cell culture monolayers from treated cells were stained with the tetrazolium dye XTT to evaluate the cellular viability of the Huh-luc/neo-ET reporter cell line incubated in the presence of the compounds. XTT-tetrazolium is metabolized by the mitochondrial enzymes of metabolically active cells to a soluble formazan product, allowing rapid quantitative analysis of the cell killing by the test substances. XTT solution was freshly prepared as a stock of I mg/ml in PBS. Phenazine methosulfate (PMS) solution was prepared at 0.15 mg/ml in PBS and stored in the dark at −20° C. until used. XTT/PMS solution was prepared immediately before use by adding 40 μL of PMS per ml of XTT solution. Fifty microliters of XTT/PMS was added to each well of the plates, adhesive plate sealers were used to seal the plates and the plates were incubated for 4 hours at 37° C. in an environment of 5% CO₂. The sealed plates were inverted several times to mix the soluble formazan product and then read spectrophotometrically at 450 nm with a Molecular Devices Spectramax 384 plus plate reader. The data were processed by the supporting SoftMax Pro 5.4.2 software and imported into a Prichard and Shipman MacSynergy II software template for analysis. The data for the single compound IFNa2b antiviral control were imported into a customized Microsoft Excel workbook for determination of the 50% cytotoxicity concentration (TCso). All cytotoxicity values were normalized to the amount of formazan in the untreated cell controls and subtracted from a background colorimetric control.

### Data Analysis:

Raw data was collected from the Wallac 1450 Microbeta Trilux liquid scintillation counter and Softmax Pro 5.4.2 software were imported into the Prichard and Shipman MacSynergy II software template (Antiviral Research 14: 181-206 [1990]). Effects of the drug combination are calculated based on the activity of the two compounds when tested alone. The expected additive antiviral protection is subtracted from the experimentally determined antiviral activity at each combination concentration resulting in a positive value (synergy, or potentiation), a negative value (antagonism), or zero (additivity). The results of the combination assays are presented three dimensionally at each combination concentration, yielding a surface of activity extending above (synergy) or below (antagonism) the plane of additivity. The volume of the surface is calculated and expressed as a synergy volume (μM²%) calculated at the 95% confidence interval. Results are expressed as the median (±standard deviations) (3 experiments) or individual values (1 or 2 experiments).

### Combination Therapy Evaluations:

SPC3649 was evaluated in combination with six known anti-HCV agents for the inhibition of HCV genotype Ib replicon in Huh-luc/neoET cells. The experimental design utilized a checkerboard dilution matrix of two-fold or five-fold serial dilutions of each of the compounds. Analysis also included wells in which the compounds were used individually or in which no compound was added.

Analysis for all combinations were initially performed using a 1:5 dilution scheme for the control compounds with a high test concentration of two times the calculated EC₅₀ of the control compound. Results of these analyses were determined to be valid only if two or more of the concentrations for each compound alone fell on the dose-response curve and if the single compound curve demonstrated a dose-dependent response in antiviral activity. Replicate analysis was performed using a 1:2 dilution scheme for the control compounds with a high test concentration of two times the calculated EC₅₀ of the control compound. The theoretical additive interactions of the two compounds were determined from the dose-response curves of the individual compounds using the MacSynergy II program which calculates the theoretical additive interactions of the drugs based on the Bliss Independence mathematical definition of expected effects for drug-drug interactions. The anti-HCV efficacy and cytotoxicity synergy volumes for the combination of SPC 3649 with each of the anti-viral compounds was calculated at the 95%, confidence interval. The results are expressed as the median (±standard deviations) (>3 experiments) or individual values (1 or 2 experiments) for the assays that met the acceptance criteria and are summarized in Table B. The synergy and antagonism volumes for three of four independent replicates of SPC 3649 with ribavirin ranged from 4.2 to 25.9 μM²% and −3.1 to −31.8 μM²%, respectively, indicating an additive interaction. SPC 3649 in combination with ribavirin resulted in a highly antagonistic interaction in a fourth experiment, with an antagonism volume of −292.6 μM²%. The overall interaction of SPC 3649 with ribavirin had a median synergy and antagonism volume for the four replicates of 4.20 μM²% and −24.2 μM²%, respectively.

TABLE B
Antiviral activity and cytotoxicity of miravirsen in combination
with other anti HCV therapeutics.

| | (Synergy vol (μM² %)/Antagonism vol | | |
|---|---|---|---|
| | (μM² %)) ± SD¹ | | |
| Second Drug | Efficacy | Cytotoxicity | Interaction |
| Interferon alpha-2b | 10.75/−38.2; 10.33/0 | 0/0; 0/−8.9 | Positive |
| Ribavirin | 4.2/−2.42(±11.70/138.22) | 0/0 | Positive |
| VX-222 | 4.81/−34.44; 0.82/−4.27 | 0/−28.6; 0/0 | Positive |
| BMS 790052 | 0/−24.5; 18.46/−0.06 | 0/−21.9 | Positive |
| Telaprevir | 0/−3.2 (±3.08/6.89) | 0/−5.05 | Positive |
| 2′-C-Methylcytidine | 0/−27.6 (±1.33/38.01) | 0/−0.67 | Positive |
| | | (±_27.36) | |

¹Volume of synergy or antagonism (μM² %) were calculated according to the method of Prichard and Shipman at the 95% confidence interval; results represent the median (±SD) (≧3 experiments) or individual values (1 or 2 experiments).

SPC3649 in combination with NS5B polymerase non-nucleoside VX-222 resulted in synergy volumes of 4.8 and 0.8 μM²%, and antagonism volumes of −34.4 and −4.27 μM²%.

The synergy volumes for two replicate assays for the combination of SPC 3649 with the NS5A inhibitor BMS 790052 were 0 and 18.5 μM²% and antagonism volumes of −25.5 and −0.1 μM²%.

The combination of SPC 3649-09 with the NS3 protease inhibitor telaprevir also indicated a positive interaction for three replicate assays with median mean synergy and antagonism volumes of 0.00 μM²% and −3.22 μM²%, respectively.

SPC3649 in combination with NS5B polymerase nucleoside inhibitor 2-methyl cytidine resulted in positive interactions for two of the assay replicates and a slightly antagonistic interaction for a third replicate. The median synergy and antagonism volumes for the combination of SPC 3649 and 2-methyl cytidine were 0.0 μM²% and −27.6 μM²%, respectively, yielding a positive interaction from the mean of three qualified assay replicates. Evaluation of the impact of combination on in vitro cytotoxicity revealed little to no cytotoxicity for each drug alone or in combination.

### Example 3

### In Vitro Antiviral Activity of Miravirsen (SPC3649) Against Wild-Type and Drug-Resistant HCV Genotype 1 b Replicons

The objective of this study was to evaluate the in vitro antiviral activity of miravirsen (SPC3649) against wild-type HCV genotype 1b replicon and NS3, NS5A and NS5B drug-resistant HCV genotype 1 b replicons in transient transfection assays utilizing Huh 7 cells.

### Method:

The in vitro antiviral of miravirsen was evaluated against wild-type HCV genotype 1 b replicon and HCV genotype 1 b replicons constructed to contain key amino acid substitutions in NS3 protease (A156T, R155K), NS5B polymerase (S282T, M423I) and NS5A protein (Y93H) in transient transfection assays utilizing Huh 7 cells. Five reference compounds (BMS-790052 (NS5A), VX-222 (NS5B), telaprevir (NS3), BILN-2061(NS3) and 2′Me-C (NS5B)) were included as drug-resistant controls. Huh 7 cells were transfected with either the wild-type or the mutant RNA constructs by electroporation. Luciferase activity was measured 72 hours after compound treatment and EC₅₀ values determined from dose-response curves. Fold resistance was expressed as the ratio of the EC₅₀ for mutant HCV replicon to the EC₅₀ for wild-type HCV replicon.

### Results:

HCV replicons constructed to contain mutations demonstrated resistance distinct for each drug class tested (Table C). Specifically, HCV replicons with amino acid substitutions A156T and R155K in NS3 were 36.1 and 4.6 fold-resistant respectively, to the protease inhibitor telaprevir; a replicon with S282T in NS5B was 42.8 fold-resistant to the NS5B nucleoside inhibitor 2′Me-C; a replicon with M423I in NS5B was 4.4 fold-resistant to the NS5B non-nucleoside inhibitor VX-222; and a replicon with Y93H in NS5A was 29.9 fold-resistant to the NS5A inhibitor BMS 790052. In contrast, miravirsen demonstrated broad activity against all drug-resistant HCV variants tested with fold changes in resistance less than 2-fold.

The in vitro antiviral of miravirsen was evaluated against wild-type HCV genotype 1b replicon and HCV replicons constructed to contain key amino acid substitutions in NS3 protease (A156T, R155K), NS5B polymerase (S282T, M423I) and NS5A protein (Y93H) in transient transfection assays. Five reference compounds (BMS-790052 (NS5A), VX-222(NS5B), telaprevir (NS3), BILN-2061(NS3) and 2′Me-C (NS5B)) were included as drug-resistant controls. Huh 7 cells were transfected with either the wild type or the mutant RNA constructs by electroporation. Luciferase activity was measured 72 hours after compound treatment and EC50 values determined from dose-response curves. Fold resistance was expressed as the ratio of the EC₅₀ for mutant HCV replicon to the EC₅₀ for wild-type HCV replicon. Results are expressed as the mean of two individual experiments. In an initial set of experiments, the standard protocol was modified by adding compounds 24 hours after electroporation. Experiments were repeated utilizing a standard protocol to maximize exposure of cells transfected with RNA constructs that impart varying levels of HCV replication fitness and to reduce the observed interexperimental variability. In a third experiment cells were pretreated with miravirsen for 24 hours before electroporation with wild-type RNA constructs to determine if preincubation could result in increased miravirsen antiviral activity.

HCV replicons constructed to contain mutations demonstrated resistance distinct for each drug class tested. Specifically, HCV replicons with amino acid substitutions A156T and R155K in NS3 were 36.1 and 4.6 fold-resistant respectively, to the protease inhibitor telaprevir; a replicon with S282T in NS5B was 45.8 fold resistant to the NS5B nucleoside inhibitor 2′Me-C; a replicon with M423I in NS5B was 4.4 fold-resistant to the NS5B non-nucleoside inhibitor VX-222; and a replicon with Y93H in NS5A was 29.9 fold-resistant to the NS5A inhibitor BMS 790052. In contrast, miravirsen demonstrated broad activity against all drug-resistant HCV variants tested with fold changes in resistance less than 2-fold.

TABLE C
Antiviral susceptibilities of HCV genotype 1b replicon containing
mutations in NS3, NS5B and NS5A to miravirsen.

| | Fold Resistance^{a} | | | | | | | | | |
|---|---|---|---|---|---|---|---|---|---|---|
| Compound | NS3 A156T | | NS3 R155K | | NS5B S282T | | NS5B M423I | | NS5A Y93H | |
| miravirsen | 1.0 | (1.4, 0.5) | 1.2 | (1.9, 0.4) | 1.9 | (2.6, 1.1) | 1.7 | (2.3, 1.0) | 0.9 | (0.8, 0.9) |
| Telaprevir | 36.1 | (46.9, 25.2) | 4.6 | (5.0, 4.2) | 1.0 | (1.0, 1.0) | NT | | NT | |
| 2′Me-C | 0.9 | (1.0, 0.8) | 0.8 | (1.0, 0.6) | 45.8 | (49.3, 42.3) | 1.0 | (0.9,1.1) | 1.0 | (1.2, 0.8) |

| | | | | | | |
|---|---|---|---|---|---|---|
| VX-222 | NT | NT | NT | 4.4 | (3.6, 5.2) | NT |

| | | | | | | |
|---|---|---|---|---|---|---|
| BMS 790052 | NT | NT | NT | NT | 29.9 | (32.7, 27.0) |

Results represent the mean fold change in susceptibility from two experiments (individual values); NT=not tested. During the conduct of these studies, the antiviral activity of miravirsen against wild-type HCV in transient transfection assays (average EC₅₀ of 36.7 μM;) were observed to be reduced when compared to antiviral activity previously reported against stable HCV cell lines (average EC₅₀ of 0.671 μM). To determine if the time of miravirsen addition could increase the relative antiviral activity, Huh 7 cells were preincubated with miravirsen 24 hours before electroporation with wild-type RNA constructs. Preincubation of cells with miravirsen did not however impact the antiviral activity (EC50 of 25.6 μM).

### Conclusions:

HCV replicons constructed to contain key amino acid substitutions in NS3 protease (A156T, R155K), NS5B polymerase (S282T, M423I) and NS5A protein (Y93H) demonstrated resistance distinct for each drug class tested. In contrast, miravirsen demonstrated broad antiviral activity against HCV replicons resistant to NS3, NS5A and NS5B inhibitors.

### Example 4

### Selection of SPC3649-Resistant HCV-1 b Replicon Cells

This example summarises the results of the in vitro selection of SPC3649 resistant HCV replicon cells. The reporter cell line Huh-luc/neo-ET was cultured under selective pressure in the presence of G418 and four independent fixed concentrations of SPC3649 or the NS3 protease inhibitor telaprevir. Control cultures included replicon cells cultured under G418 selective pressure in the absence of compound or in the presence of scrambled oligonucleotide control SPC4729. Selection in the presence of SPC3649 resulted in a decrease in the rate of cell expansion, but failed to produce distinct individual resistant clonal populations. Selection in the presence of telaprevir resulted in a decrease in the rate of cell expansion, and the production of distinct individual clonal populations. Selection in the absence of compound or the presence of SPC4729 did not decrease the rate of cell expansion.

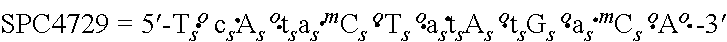

Cell culture and Assay setup: The reporter cell line Huh-luc/neo-ET was obtained and prepare as described previously. Cells were plated into 10 cm tissue culture dishes at a density of 3×10⁵ cells per plate in culture medium containing 250 μg/ml G418 and incubated for 24 hours at 37° C. in an environment of 5% CO₂. Following the 24 hour incubation, dilutions of SPC3649, SPC4729, or telaprevir were prepared in cell culture medium (as above) with 750 μg/ml G418. Each of the dilutions was added to duplicate plates and the plates were incubated at 37° C. in an environment of 5% CO². Two additional plates received cell culture medium with 750 μg/ml G418 as a no-treatment control. The final SPC3649 concentrations were 1.00 μM, 2.50 μM, 5.00 μM, and 10.0 μM (2×, 5×, 10×, and 20× the EC₅₀ concentration, respectively). The final telaprevir concentrations were 0.600 μM, 1.50 μM, 3.00 μM, and 6.00 μM (2×, 5×, 10×, and 20× the EC₅₀ concentration, respectively). The final concentration of SPC4729 was 10.0 μM. The cells were split at ratios of 1:4 to 1:3 bi-weekly for 28 days or until there was an observable reduction in cell growth rate as determined by the confluence of the cells in the culture dishes. Once the growth rate decreased, cells were spilt at a ratio of 1:2 or medium was replenished with fresh medium without splitting the cells. Following 28 days of passage, one set of dishes was stained with crystal violet and the other set of plates was utilized for expansion and establishment of resistant cell culture stocks.

### Results:

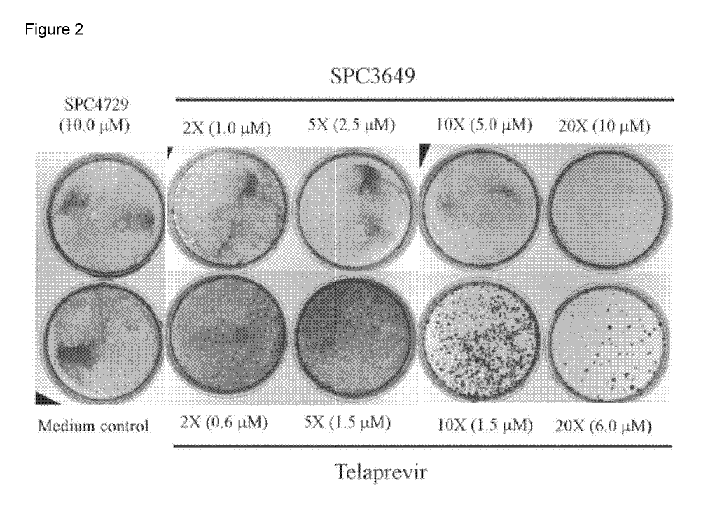

Huh-luc/neo-ET HCY genotype Ib replicon cells were cultured under selective pressure in the presence of 750 μg/mL G4 I8 with and without four independent fixed concentrations of SPC3649 anti-miR oligonucleotide, or telaprevir, or one concentration of scrambled oligonucleotide SPC4729 for 28 days in tissue culture plates. Duplicate tissue culture dishes were established and maintained for each culture condition. Medium was replaced bi-weekly during the 28 days of culture and the cells were split at the time of medium replenishment to maintain a confluent cell culture monolayer. Following four weeks in culture, cells were either fixed with methanol and stained with crystal violet or expanded for subsequent phenotypic and genotypic characterization. Cells cultured in the presence of 750 μg/ml G418 alone or 750 μg/ml G418 with 10.0 μM of scrambled oligonucleotide SPC4729 required bi-weekly splits at ratios of 1:3 or 1:4 in fresh medium (Table D). Culture of cells in the presence of 750 μg/ml G418 and SPC3649 resulted in a decrease in the rate of cell expansion as indicated by a dose-dependent decrease in the required dilution of the cells during medium exchange and passage (Table E); however, distinct individual resistant clonal populations were not produced as a result of SPC3649 selection (FIG. 2). Selection in the presence of telaprevir resulted in a dose-dependent decrease in the rate of cell expansion and the production of distinct individual resistant clonal populations (Table F and FIG. 2).

TABLE D
Passage of cells in the presence of G418 alone or G418 plus
control oligo SPC4729: Cell split ratios.

| | Medium plus | SPC 4726-03 |
|---|---|---|
| Day | G-418 | 10.0 μM |
| 4 | 1:3 | 1:3 |
| 8 | 1:3 | 1:3 |
| 11 | 1:3 | 1:3 |
| 15 | 1:4 | 1:4 |
| 18 | 1:4 | 1:4 |
| 22 | 1:4 | 1:4 |
| 25 | 1:4 | 1:4 |

TABLE E
Passage of cells in the presence of G418 alone or G418 plus the indicated
concentration of SPC3649: Cell Split ratios.

| Day | 1.00 μM | 2.50 μM | 5.00 μM | 10.0 μM |
|---|---|---|---|---|
| 4 | 1:3 | 1:3 | 1:3 | 1:3 |
| 8 | 1:3 | 1:3 | 1:3 | 1:3 |
| 11 | 1:3 | 1:3 | 1:2 | 1:2 |
| 15 | 1:3 | 1:2 | 1:2 | 1:2 |
| 18 | 1:3 | 1:3 | 1:2 | 1:2 |
| 22 | 1:4 | 1:2 | 1:3 | 1:3 |
| 25 | 1:3 | 1:3 | 1:2 | 1:2 |

TABLE F
Passage of cells in the presence of G418 alone or G418 plus the indicated
concentration of NS3 protease inhibitor telaprevir: Cell split ratios.

| Day | 0.600 μM | 1.50 μM | 3.00 μM | 6.00 μM |
|---|---|---|---|---|
| 4 | 1:3 | 1:3 | 1:3 | 1:3 |
| 8 | 1:3 | 1:3 | 1:3 | 1:3 |
| 11 | 1:3 | 1:2 | 1:2 | N.S. |
| 15 | 1:2 | N.S. | N.S. | N.S. |
| 18 | 1:2 | 1:3 | N.S. | N.S. |
| 22 | 1:3 | N.S. | N.S. | N.S. |
| 25 | 1:4 | 1:4 | N.S. | N.S. |

N.S. = not split. On these days media was changed, but the cell were not split.

### Example 5

### Anti-HCV Evaluations of Ribavirin in Combination with Telaprevir or Scrambled Oligonucleotide SPC 4729 in HCV Genotype-Ib Replicon Cells

This example summarizes the results of the in vitro evaluation of ribavirin in combination with NS3/4A protease inhibitor telaprevir, or a scrambled oligonucleotide. The combination antiviral assays were performed using the reporter cell line Huhluc/neo-ET, which contains a bicistronic HCV genotype Ib replicon. Combination data were analyzed using MacSynergy II software at the 95%, confidence interval. The in vitro combination assays were designed to define the antiviral interaction of the two compounds and to determine if their interaction was synergistic or antagonistic.

The reporter cell line Huh-luc/neo-ET was obtained and prepare as described previously. The cells were treated with trypsin and enumerated by staining with trypan blue and seeded into 96-well tissue culture plates at a cell culture density 5.0×10³ cells per well in a volume of 85 μl per well and incubated at 37° C. in an environment of 5% CO₂ for 24 hours. Six plates were established for the determination of combination efficacy (EC₅₀) and cytotoxicity (TC₅₀) for each of the compound combinations (three plates each for efficacy and toxicity). Following the 24 hour incubation, 2-fold serial dilutions of each of the compounds were prepared in cell culture medium without G418 and added to the wells in a checkerboard pattern. For the ribavirin plus telaprevir combination analysis, eight 2-fold serial dilutions of ribavirin (concentration range of 148 μM to 1.16 μM) and five 2-fold dilutions of telaprevir (concentration range of 1.00 μM to 62.5 nM) were added to the cells. For the SPC 4729 plus ribavirin combination analysis, eight 2-fold serial dilutions of SPC 4729 (concentration range of 2.4 μM to 18.8 nM) and five 2-fold dilutions of ribavirin (concentration range of 148 μM to 9.25 nM) were added to the cells. Six wells in each plate also received medium alone as a no-treatment control. Separate duplicate plates were prepared in which IFN-a2b was added to triplicate wells at final concentrations of 10.0, 2.00, 0.400, 0.0800, 0.0160, and 0.0032 units per milliliter (U/mL) as a positive single compound control for antiviral efficacy. The cells were incubated for 48 hours at 37° C. in an environment of 5% CO₂. Following incubation, the plates were assessed for anti-HCV activity by measurement of luciferase reporter activity and cellular cytotoxicity by XTT staining.

### Measurement of Virus Replication, Cytotoxicity and Data Analysis:

As per Example 2

### Combination Therapy Evaluations:

Ribavirin was evaluated in combination with telaprevir and scrambled oligonucleotide SPC 4729-03 for the inhibition of HCY genotype Ib replicon in Huh-luc/neo-ET cells. The experimental design utilized a checkerboard dilution matrix of two-fold serial dilutions of each of the compounds. Analysis also included wells in which the compounds were used individually or in which no compound was added. Three independent experiments was performed for each of the combinations. The theoretical additive interactions of the two compounds were determined from the dose-response curves of the individual compounds using the MacSynergy II program which calculates the theoretical additive interactions of the drugs based on the Bliss Independence mathematical definition of expected effects for drugdrug interactions. The anti-HCV efficacy and cytotoxicity synergy volumes for the combinations were calculated at the 95% confidence level. Results for analysis of ribavirin plus telaprevir combination were determined to be valid only if two or more of the concentrations for each compound alone fell on the dose-response curve and if the single compound curve demonstrated a dose-dependent response in antiviral activity or cytotoxicity. Results for the ribavirin plus SPC 4729 combination were determined to be valid if two or more of the ribavirin concentrations fell on the dose-response curve, if the ribavirin single compound curve demonstrated a dose-dependent response in antiviral activity and no antiviral activity was observed for SPC 4729. The results summarized in Table F are expressed as individual values of two independent experiments for the ribavirin plus telaprevir combination and a single value for the ribavirin plus SPC 4729 combination.

TABLE F
Results of combination analysis of ribavirin with telaprevir or SPC 4729

| | Efficacy | Cytotoxicity |
|---|---|---|
| Ribavirin | (μM² %[Synergy vol/ | (μM² %[Synergy vol/ |
| Plus | Antag Vol]) ±SD | Antag Vol]) ±SD |
| Telaprevir | 37.5/22.6; | 0.00/−50.9 |
| | 0.00/−200.2 | |
| SPC 4729-03 | 0.00/−177.6 | 0.00/−44.3 |
| | | (±0.82/171) |

The overall interaction of ribavirin and telaprevir were inconclusive. The interaction of ribavirin and the scrambled control SPC 4729 was antagonistic, in contrast to the data for SPC3649 (Example 2), indicating an apparent synergy between SPC3649 and ribavirin. Evaluation of the impact of combination on in vitro cytotoxicity revealed an antagonistic interaction for ribavirin plus telaprevir (reduction in overall cytotoxicity) and the interaction for ribavirin plus SPC 4729 slight higher cytotoxicity.

### Example 6

### Phase 2a Clinical Trail—Miravirsen Monotherapy

### Background:

Hepatitis C virus (HCV) replication is dependent on a functional interaction between host microRNA-122 (miR-122) and the HCV genome. Miravirsen is a β-D-oxy-Locked Nucleic Acid (LNA)-modified phosphorothioate antisense oligonucleotide that targets liver-specific miR-122. Miravirsen demonstrated activity against all HCV genotypes in vitro and long-lasting HCV RNA suppression without emergence of resistance in vivo. This ascending multiple dose phase IIa study evaluated the safety and efficacy of miravirsen in chronic HCV infected patients.

### Methods:

Thirty-six treatment-naïve chronic HCV genotype 1 infected patients were enrolled into 3 sequential dosing cohorts and received 5 weekly doses of miravirsen 3, 5, or 7 mg/kg (n=27) or placebo (n=9) subcutaneously over 29 days and were followed up until week 18.

### Results:

Miravirsen resulted in a dose-dependent reduction in HCV RNA that was prolonged well beyond the end of active therapy. The mean of the maximum HCV RNA decline from baseline was 1.2 (p=0.011), 2.9 (p=0.003) and 3.0 (p=0.002) log₁₀ IU/mL in the 3, 5, and 7 mg/kg miravirsen treated cohorts, respectively. This decline was 0.4 log₁₀ IU/mL in the placebo cohort. One patient who received 5 mg/kg and four patients who received 7 mg/kg achieved undetectable HCV RNA. There were no dose-limiting adverse events and no escape mutations were observed in the HCV genome miR-122 binding sites.

### Conclusions:

Miravirsen is the first microRNA-targeted therapy to be administered to patients. In chronic HCV genotype 1 infected patients, miravirsen was safe, well-tolerated, and resulted in prolonged dose-dependent reductions in HCV RNA levels. Emergence of viral resistance to miravirsen was not seen. (ClinicalTrials.gov number NCT01200420).

1. A miR-122 inhibitor for use in the treatment of Hepatitis C (HCV) in combination with an HCV NS3/4A protease inhibitor, and optionally ribavirin (or a virally active derivative thereof).

2. The miR-122 inhibitor according to claim 1 or 2, wherein the miR-122 inhibitor is an oligomer which consists or comprises the formula:
5′-^{m}Cₛ^{o}cₛAₛ^{o}tₛtₛGₛ^{o}Tₛ^{o}cₛaₛ^{m}Cₛ^{o}aₛ^{m}Cₛ^{o}tₛ^{m}Cₛ^{om}C^{o}-3′
wherein; a lowercase letter identifies a DNA unit, and an upper case letter identifies a LNA unit, ^{m}C identifies a 5-methylcytosine LNA, subscript ₛ identifies a phosphorothioate internucleoside linkage, and wherein LNA units are beta-D-oxy, as identified by a ^{o} superscript after LNA residue.

3. The miR-122 inhibitor according to claim 1 or 2, wherein the HCV NS3/4A protease inhibitor is selected from the group consisting of Tegobuvir, Narlaprevir, Vaniprevir, Danoprevir, Filibuvir, Telaprevir, Coceprevir and Boceprevir; ABT-450 (Abbott/Enanta), ACH-2684; ACH-1625 (Achillion), BMS-650032 (BMS); BMS-791325 (BMS), TMC435 (Tibotec), GS-9256 (Gilead), RG7227 (Intermune/Genetech), MK-5172 (Merck); MK-7009 (Merck), and BI 201335 (Boehringer Ingleheim Pharma).

4. The miR-122 inhibitor according any one of claims 1-2, wherein the HCV NS3/4A protease inhibitor is telaprevir or boceprevir.

5. The miR-122 inhibitor according to any one of claims 1-4 wherein the treatment is interferon free.

6. The miR-122 inhibitor according to any one of claims 1-5, wherein the treatment further comprises the use of a direct acting agent selected from the group consisting of an HCV NS5A protein inhibitor, and a HCV NS5B polymerase inhibitor (such as a non-nucleoside inhibit and/or a nucleoside inhibitor).

7. The miR-122 inhibitor according to any one of claims 1-5, wherein the combined treatment of the miR-122 inhibitor and the HCV NS3/4A protease inhibitor occurs in a combination treatment period of less than 1 year, such as 4-48 weeks.

8. The miR-122 inhibitor according to claim 7 wherein the duration of the combination treatment period, is less than 48 weeks, such as less than 24 weeks, or less than 13 weeks.

9. The miR-122 inhibitor according to claim 7 or 8, wherein the combination treatment period is preceded with a pre-treatment period of the miR-122 inhibitor optionally in combination with ribavirin or a virally active derivative thereof, and in the absence of the HCV NS3/4A polymerase inhibitor.

10. The miR-122 inhibitor according to claim 9, wherein the pre-treatment period is of 1-12 weeks in duration.

11. The miR-122 inhibitor according to any one of claims 1-10, wherein the subject is an non responder, a partial responder, a relapse responder or a null responder or a non-responder to interferon or a direct acting agent, such as an inhibitor of HCV NS5A protein, or an inhibitor of HCV NS5B polymerase.

12. The use of a miR-122 inhibitor for the preparation of a medicament for the treatment of Hepatitis C, wherein said medicament is for use in combination with an HCV NS3/4A protease inhibitor.

13. A method for the treatment of hepatitis C (HCV) infection in a subject infected with HCV, said method comprising the steps of administering an effective amount of a miR-122 inhibitor and an effective amount of a HCV NS3/4A protease inhibitor to the subject infected with HCV.

14. The method according to claim 13, wherein said method further comprises administering an effective amount of ribavirin, or a virally active derivative thereof to the subject.

15. The method according to claim 13 or 14, wherein the oligomer consists or comprises the formula:
5′-^{m}Cₛ^{o}cₛAₛ^{o}tₛtₛGₛ^{o}Tₛ^{o}cₛaₛ^{m}Cₛ^{o}aₛ^{m}Cₛ^{o}tₛ^{m}Cₛ^{om}C^{o}-3′
wherein; a lowercase letter identifies a DNA unit, and an upper case letter identifies a LNA unit, ^{m}C identifies a 5-methylcytosine LNA, subscript ₛ identifies a phosphorothioate internucleoside linkage, and wherein LNA units are beta-D-oxy, as identified by a ^{o} superscript after LNA residue.

16. The method according to any one of claims 13-15, wherein the HCV NS3/4A protease inhibitor is selected from the group consisting of Tegobuvir, Narlaprevir, Vaniprevir, Danoprevir, Filibuvir, Telaprevir, Coceprevir and Boceprevir; ABT-450 (Abbott/Enanta), ACH-2684; ACH-1625 (Achillion), BMS-650032 (BMS); BMS-791325 (BMS), TMC435 (Tibotec), GS-9256 (Gilead), RG7227 (Intermune/Genetech), MK-5172 (Merck); MK-7009 (Merck), and BI 201335 (Boehringer Ingleheim Pharma).

17. The method according any one of claims 13-15, wherein the HCV NS3/4A protease inhibitor is telaprevir or boceprevir.

18. The method according to any one of claims 13-17 wherein the treatment is interferon free.

19. The method according to any one of claims 13-18, wherein multiple doses of the miR-122 inhibitor and multiple doses of the HCV NS3/4A protease inhibitor, and optionally ribavirin (or a virally active derivative thereof) are administered over a combination treatment period of less than 1 year, such as 4-48 weeks.

20. The method according to claim 19 wherein the duration of the treatment period, is less than 48 weeks, such as less than 24 weeks, or less than 13 weeks.

21. The method according to claim 19 or 20, wherein the combination treatment period is preceded with a pre-treatment period of the miR-122 inhibitor, optionally in combination with ribavirin (or a virally active derivative thereof).

22. The method according to any one of claims 13-21, wherein the subject is an non responder, a partial responder, a relapse responder or a null responder or a non-responder to interferon or a direct acting agent, such as an inhibitor of HCV NS5A protein, or an inhibitor of HCV NS5B polymerase.


SEQUENCE LISTING (4 sequences)

SEQ ID NO: 1 - RNA, 22 nt, homo sapiens
uggagugugacaaugguguuug

SEQ ID NO: 2 - DNA, 15 bp, artificial
  Miravirsen nucleobase sequence
ccattgtcacactcc

SEQ ID NO: 3 - DNA, 15 bp, artificial
  Scambled control oligo nucleobase sequence
tcatactatatgaca

SEQ ID NO: 4 - RNA, 85 nt, homo sapiens
ccuuagcagagcuguggagugugacaaugguguuugugucuaaacuaucaaacgccauuaucacacuaaauagcuacugcuaggc